(19) United States
(12) Patent Application Publication
(10) Pub. No.: US 2007/0092526 A1
(43) Pub. Date: Apr. 26, 2007

(54) Polynucleotide vaccine adjuvants and formulations containing cationic surfactants, and methods of use

(76) Inventor: Robert K. Evans, Souderton, PA (US)

Correspondence Address: MERCK AND CO., INC, P O BOX 2000, RAHWAY, NJ, 07065-0907, US

(21) Appl. No.: 09/888,304

(22) Filed: Jun. 22, 2001

Related U.S. Application Data
(60) Provisional application No. 60/214,824, filed on Jun. 28, 2000.
(60) Provisional application No. 60/213,622, filed on Jun. 23, 2000.

(51) Int. Cl. A61K 48/00; A61K 39/00

(52) U.S. Cl. 424/184.1; 514/44

(57) ABSTRACT

Improved polynucleotide vaccine adjuvants and related polynucleotide vaccine formulations are disclosed which are useful in prophylactic or therapeutic vaccine and/or gene therapy-based applications. These adjuvants comprise a block copolymer and a cationic surfactant component. The inclusion of a cationic surfactant results in an increased percentage of polynucleotide that is physically associated with the adjuvant in vitro, resulting in enhanced in vivo immune responses to polynucleotide vaccines.

### CROSS-REFERENCE TO RELATED APPLICATIONS

This application claims the benefit, under 35 U.S.C. 119(e), of U.S. Provisional Application Ser. No. 60/214,824 filed Jun. 28, 2000 and U.S. Provisional Application Ser. No. 60/213,622 filed Jun. 23, 2000, both of which are hereby incorporated in their entirety.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to polynucleotide vaccine adjuvants, formulations comprising a polynucleotide and adjuvant, related pharmaceutical products and methods of using these formulations and pharmaceutical products in prophylactic or therapeutic vaccine and/or gene therapy-based applications. A preferred formulation disclosed herein is an adjuvanted polynucleotide vaccine formulation which comprises a polynucleotide component and adjuvant component, the adjuvant component comprising a non-ionic block copolymer and a cationic surfactant. The inclusion of a cationic surfactant results in an increased percentage of polynucleotide that is physically associated with the adjuvant in vitro. To this end, these adjuvanted formulations provide a significant enhancement of in vivo immune responses to polynucleotide vaccines and/or gene therapy-based transgenes when compared to known polynucleotide vaccine adjuvants.

### BACKGROUND OF THE INVENTION

Alternative forms of immunization for DNA vaccines have been reported to increase the immunogenicity of encoded antigens. These approaches are well known in the art and include particle bombardment using DNA-coated gold beads, co-administration of DNA vaccines with plasmid DNA expressing cytokines, chemokines or costimulatory molecules, formulation of DNA with cationic lipids or with experimental adjuvants such as monophosphoryl lipid A, as well as co-administration of a DNA vaccine with certain mineral salts, such as aluminum- or calcium-based adjuvants. The enhancement of DNA vaccine potency is abrogated if the DNA was physically bound to the aluminum adjuvant, a distinct difference from conventional vaccines in which protein antigens are generally bound to “alum” (See PCT/US98/02414, WO 98/35562). Newman et al. (1998, Critical Reviews in Therapeutic Drug Carrier Systems 15(2): 89-142) review a class of non-ionic block copolymers which show adjuvant activity. The basic structure comprises blocks of polyoxyethylene (POE) and polyoxypropylene (POP) such as a POE-POP-POE block copolymer. Newman et al. id., disclose that certain POE-POP-POE block copolymers may be useful as adjuvants to an influenza protein-based vaccine, namely higher molecular weight POE-POP-POE block copolymers containing a central POP block having a molecular weight of over about 9000 daltons to about 20,000 daltons and flanking POE blocks which comprise up to about 20% of the total molecular weight of the copolymer (see also U.S. Reissue Pat. No. 36,665, U.S. Pat. No. 5,567,859, U.S. Pat. No. 5,691,387, U.S. Pat. No. 5,696,298 and U.S. Pat. No. 5,990,241, all issued to Emanuele, et al., regarding these POE-POP-POE block copolymers). WO 96/04932 further discloses higher molecular weight POE/POP block copolymers which have surfactant characteristics and show biological efficacy as vaccine adjuvants.

U.S. Pat. No. 5,656,611, issued Aug. 12, 1997, and WO 99/06055 disclose compositions which comprise a polynucleotide, and a block copolymer containing a non-ionic portion and a polycationic portion. Evidently, a surfactant is added to increase solubility and the end result is the formation of micelles.

WO 99/21591 discloses a soluble ionic complex comprising an aqueous mixture of a polynucleotide and a benzylammonium group-containing surfactant.

Despite these reports, there remains a need for the development of DNA vaccine adjuvants and related polynucleotide vaccine formulations which enhanoe the in vitro physical association of plasmid DNA with adjuvant, thus promoting a concomitant enhancement in in vivo immune responses. The present invention addresses and meets these needs by disclosing (1) adjuvants which show increased association with polynucleotides in vitro, and (2) polynucleotide vaccine formulations containing these adjuvants which show a concomitant increase in the predicted in vivo immune response associated with the respective polynucleotide.

### SUMMARY OF THE INVENTION

The present invention relates in part to polynucleotide vaccine adjuvants which comprise a block copolymer and a cationic surfactant. The inclusion of a cationic surfactant results in an increased percentage of polynucleotide that is physically associated with the block copolymer/cationic surfactant upon mixing and/or temperature cycling through the block copolymer cloud point, thus resulting in an enhanced in vivo immune response to polynucleotide vaccines and/or gene therapy-based transgenes. To this end, the present invention relates in part to an adjuvant for use in polynucleotide vaccine and gene therapy-based applications wherein the adjuvant has an enhanced propensity to associate with a negatively charged polynucleotide vaccine component, resulting in a greater percentage of polynucleotide within the vaccine formulation being associated with the adjuvant prior to host administration.

The present invention also relates to pharmaceutically acceptable polynucleotide vaccine formulations which comprise a polynucleotide component and adjuvant component, the adjuvant component comprising a block copolymer and a cationic surfactant. These formulations will contain a percentage of polynucleotide associated with the adjuvant and a percentage of free polynucleotide (i.e., polynucleotide which is not associated with the adjuvant in vitro). The formulations of the present invention show an increased proportion of polynucleotide associated with the adjuvant in vitro, which in turn results in a concomitant enhancement of in vivo immune responses.

Therefore, the present invention relates to adjuvant and polynucleotide vaccines comprising a non-ionic block copolymer such that the non-ionic block copolymer (1) forms microparticles ranging in size from about 100 nm to about 2000 nm, (2) has a cloud point at or below room temperature and (3) binds to cationic surfactants allowing an enhanced association of the block copolymer microparticle to nucleic acid molecules. With this in mind, the present invention relates to adjuvant and polynucleotide vaccine comprising a non-ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐ H, wherein (b) represents a number such that the molecular weight of the hydrophobic POP portion (C₃H₆O) is less than 20,000 daltons and wherein (a) represents a number such that the percentage of hydrophilic POE portion (C₂H₄O) is between approximately 1% and 40% by weight; as well as a cationic surfactant. Preferred formulations are described in detail herein.

The present invention also relates to an adjuvant which comprises a block copolymer, a cationic surfactant as described within this specification and a non-ionic surfactant, such as polysorbate-80, which may be a useful excipient to control particle aggregation in the presence of the antigen, such as a polynucleotide. Therefore, the present invention relates to an adjuvant comprising a non-ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐ H, wherein (b) represents a number such that the molecular weight of the hydrophobic POP portion (C₃H₆O) is less than approximately 20,000 daltons and wherein (a) represents a number such that the percentage of hydrophilic POE portion (C₂H₄O) is between approximately 1% and 40% by weight; a cationic surfactant and a non-ionic surfactant or other excipients to inhibit particle aggregation in the presence of a population of polynucleotide molecules.

The present invention also relates to a polynucleotide vaccine formulation which first comprises a polynucleotide, an adjuvant component comprising a block copolymer and a cationic surfactant, as described within this specification, and secondly comprising a non-ionic surfactant, such as polysorbate 80, or other excipients, such as glycerol, which may be a useful excipient to control particle aggregation in the presence of the polycation. Therefore, the present invention relates to a polynucleotide vaccine comprising a non-ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐ H, wherein (b) represents a number such that the molecular weight of the hydrophobic POP portion (C₃H₆O) is less than approximately 20,000 daltons and wherein (a) represents a number such that the percentage of hydrophilic POE portion (C₂H₄O) is between approximately 1% and 40% by weight; a cationic surfactant and a non-ionic surfactant or other excipients useful to inhibit particle aggregation in the presence of a population of polycation molecules.

The present invention also relates to methods of generating an immune response or promote expression of a therapeutic protein in a vertebrate host by administering to the host the adjuvanted polynucleotide vaccine formulations of the present invention. The preferred host is a mammalian host, preferably a human or a non-human mammal of commercial or domestic veterinary importance.

The present invention thus relates to adjuvants, polynucleotide vaccine compositions/formulations, and their respective methods of use, which are useful in promoting an effective immune response upon administration within a target host, such as a human or non-human mammal. The term “adjuvant” as used herein is not limited to a specific mode of action as it relates to the disclosed adjuvant compositions and formulations of the present invention. The term “adjuvant” as used herein may refer to a substance or combination of substances which nonspecifically enhances the immune response to an antigen, such as an antigen expressed from a delivered polynucleotide, such that expression of an antigen from the polynucleotide results in an increased in vivo immune response. Also included in this definition are substances which may act as facilitators of in vivo cellular gene delivery, thereby increasing the amount of plasmid DNA delivered to cells that can express the intended antigen. Substances which may enhance delivery of plasmid DNA would include those which do not substantially interact with the plasmid DNA in the formulation, as well as substances which do interact with the plasmid DNA, forming tightly bound or weakly bound complexes between the adjuvant and the plasmid DNA, either in vitro or in vivo.

It is an object of the present invention to provide improved adjuvants comprising a non-ionic block copolymer and a cationic surfactant, adjuvants which promote an enhanced in vivo immune response to vaccines, especially via co-administration with a polynucleotide vaccine or a polynucleotide encoding a therapeutic protein which contributes to the partial or total amelioration of a certain disease or disorder.

It is also an object of the present invention to provide for the polynucleotide-based formulations which comprise both the polynucleotide and adjuvant component as described in the previous paragraph.

It is therefore a further object of the present invention to provide for methodology to administer to the target host a polynucleotide vaccine formulation of the present invention, which in turn will promote an immune response or expression of a therapeutic protein within the target host.

It is an therefore an object of the present invention to provide adjuvants and polynucleotide vaccine formulations containing such an adjuvant that act to promote in vivo immune response in vertebrate host systems such as human and non-human mammals. As noted herein, the term adjuvant will encompass any substance or combination of substances which nonspecifically enhances the immune response to an antigen. Since this disclosure is not bound by theory, included in the definition of a substance which acts as an adjuvant would be a substance which may act as a facilitator of gene delivery into the interior of a cell, thereby increasing the amount of plasmid DNA delivered to these target cells that can express the intended antigen.

As used herein, “PBS” refers to—phosphate buffered saline—.

As used herein, “BAK” refers to—benzylalkonium chloride—.

As used herein, “BEC” refers to—benzethonium chloride—

As used herein, “CPC” refers to—scetylpyidinium chloride—.

As used herein, “CTAC” refers to—cetyl trimethylammonium chloride—.

As used herein, “Dh” refers to—hydrodynamic diameter—.

As used herein, “PS-80” refers to—polysorbate 80—.

As used herein, the words “particle” and “microparticle” are interchangeable.

As used herein, the term “adjuvant” is any substance or combination of substances which nonspecifically enhances the immune response to an antigen; and as further shown herein, also to any substance which enhances the immune response directly related to delivery of a polynucleotide within a vertebrate or mammalian host, such as a human or non-human mammalian host, such that administration of the adjuvant in combination with the polynucleotide results in an increased in vivo immune response to expression of the intended antigen or antigens encoded by the polynucleotide. Included in this definition are substances which may act as facilitators of gene delivery, thereby increasing the amount of plasmid DNA delivered to cells that can express the intended antigen. Substances which may enhance delivery of plasmid DNA would include those which do not substantially interact with the plasmid DNA in the formulation and substances which do interact with the plasmid DNA, forming tightly bound or weakly bound complexes between the adjuvant and the plasmid DNA, either in vitro or in vivo.

As used herein, the term “polynucleotide” is a nucleic acid molecule which contains essential regulatory elements such that upon introduction into a living, vertebrate cell, the nucleic acid molecule is able to direct the cellular machinery to produce translation products encoded by the genes comprising the nucleic acid molecule.

As used herein, the term “polynucleotide vaccine”, “polynucleotide vaccine formulation”, “pharmaceutical” and “vaccine” are used interchangeably to indicate polynucleotide-based compositions, including compositions which comprise the poloxamers and cationic surfactants disclosed herein, useful for inducing a prophylactic or therapeutic immune response, whether it be as a vehicle to deliver a transgene of interest to a vertebrate host, such as a human or non-human mammalian host, to promote therapeutic levels of expression of the transgene vaccine against a specific organism, or to generate both an immune response and a therapeutic level of expression.

As used herein, the term “vector” refers to a vehicle by which DNA fragments, most likely comprising a transgene or portion thereof which expresses an antigen or antigenic epitope, can be introduced into a host organism or host tissue. There are various types of vectors which include but are not limited to recombinant vectors, including DNA plasmid vectors, recombinant viral vectors such as adenovirus vectors, retrovirus vectors and adeno-associated virus vectors, as well as bacteriophage vectors and cosmid vectors.

The term “biologically effective amount” means that a sufficient amount of polynucleotide vaccine or adjuvanted polynucleotide vaccine is injected to produce the adequate levels of the polypeptide and in turn generate the required humoral and/or cellular immune response. One skilled in the art recognizes that this level may vary.

The term “gene” or “transgene” refers to a segment of nucleic acid molecule which encodes a discrete protein or a portion thereof, such as a portion of the full-length protein which will induce an appropriate immune response within the host.

### BRIEF DESCRIPTION OF THE FIGURES

FIG. 1 shows the effect of benzylalkonium and benzethonium chloride on the particle size of CRL-1005 in PBS.

FIGS. 2A and 2B show the plasmid DNA sedimentation through a 2-13% sucrose gradient at 80,000 rpm (348,000×g) for 1.5 hours. FIG. 2B has a expanded view of the y-axis. FIG. 2A is shown to indicate the amount of DNA recovered in fractions 15-16, which is not shown in FIG. 2B because of the expanded scale. All figures showing the % DNA associated to the polymer indicate the overall recovery of DNA.

FIGS. 3A and 3B shows the plasmid DNA sedimentation (FIG. 3A) and BAK sedimentation (FIG. 3B) through a sucrose gradient for 0.85 mM BAK, 5 mg/ml DNA in the absence of CRL-1005. DNA recovery was 93%.

FIGS. 4A and 4B shows the plasmid DNA sedimentation (FIG. 4A) and BAK sedimentation (FIG. 4B) through a sucrose gradient for 0.85 mM BAK, 5 mg/ml DNA sample in the presence of 7.5 mg/ml CRL-1005. DNA recovery was 97%.

FIG. 5 shows a BAK sedimentation profile for 0.85 mM BAK, 7.5 mM CRL-1005, 5 mg/ml DNA sample and 0.85 BAK alone through a 2-13% sucrose gradient prepared in PBS.

FIG. 6 shows the effect of BAK concentration on the percent plasmid DNA associated with CRL-1005.

FIG. 7 shows the reproducibility and short-term stability of formulations D118 and D121, as described in Table 2.

FIG. 8 shows the effect of BAK concentration on particle size formulations containing 22.5 mg/ml CRL-1005. The formulations of D121, D129, D130 and D131 are described in Table 2.

FIG. 9 shows the effect of 30° C. incubation on the particle size of CRL-1005 in formulations D118 and D121, as described in Table 2.

FIG. 10 shows the binding of BAK to CRL-1005 particles.

FIG. 11 shows the dissolution of DNA/BAK precipitates in formulation D118 above the cloud point of CRL-1005. Formulation D118 is described in Table 7.

FIG. 12 shows the particle size distribution (by volume %) of a formulation containing 5.0 mg/mL plasmid DNA and 0.6 mM BAK with and without CRL-1005.

FIG. 13 shows the particle size distribution (by number %) of a formulation containing 5.0 mg/mL plasmid DNA and 0.6 mM BAK with and without CRL-1005.

FIG. 14 shows the particle size distribution of CRL-1005 in formulations D113 and D118, which are described in Table 7.

FIG. 15 shows the particle size distribution of CRL-1005 in formulation D118a, which is described in Table 7.

FIG. 16 shows the particle size distribution of CRL-1005 in formulation D145, which is described in Table 7.

FIG. 17 shows the stability of the CRL-1005 particle size distribution in formulation D118a at 25° C. Formulation D118a is described in Table 7.

### DETAILED DESCRIPTION OF THE INVENTION

The present invention relates to adjuvants and the respective adjuvanted polynucleotide vaccine formulations and related pharmaceutical products for use in gene therapy and/or vaccine applications. These formulations and pharmaceutical products comprise polynucleotide molecules, a block copolymer, and a cationic surfactant. These adjuvanted nucleic acid formulations and pharmaceutical products markedly enhance the immune response of nucleic acid-based gene therapy and/or vaccine applications when compared to presently available formulations. The block copolymers useful in the DNA vaccine formulations described herein are block copolymers which form microparticles at room temperature (above the block copolymer cloud point) and have the ability to associate with a population of nucleic acid molecules, such as a population of plasmid DNA molecules, with enhanced association obtained via the addition of cationic surfactants. The nucleic acid molecule of the present invention may include a deoxyribonucleic acid molecule (DNA), such as genomic DNA and complementary DNA (cDNA) as well as a ribonucleic acid molecule (RNA). In regard to the block copolymer, it is preferable that such a block copolymer be one that possesses adjuvant capability within a DNA vaccine formulation targeted for widespread human and/or non-human animal administration. To this end, a preferred group of copolymers that will be useful in the DNA formulations of the present invention are non-ionic block copolymers which comprise blocks of polyoxyethylene (POE) and polyoxypropylene (POP). A typical POE/POP block copolymer utilized herein will comprise the structure of POE-POP-POE, as reviewed in Newman et al. (1998, Critical Reviews in Therapeutic Drug Carrier Systems 15(2): 89-142). A preferred block copolymer for use in the DNA vaccine formulations of the present invention is a POE-POP-POE block copolymer with a central POP block having a molecular weight in a range from under 1000 up to approximately 20,000 daltons and flanking POE blocks which comprise up to about 40% of the total molecular weight of the copolymer. Block copolymers such as these, which are much larger than earlier disclosed Pluronic-based POE/POP block copolymers, are described in detail in U.S. Reissue Pat. No. 36,655. A representative POE-POP-POE block copolymer utilized to exemplify DNA formulations of the present invention is disclosed in WO 96/04392, is also described at length in Newman et al. (i.d.), and is referred to as CRL 1005 (CytRx Corp). Synthesis of the non-ionic copolymers of the present invention is known and is described within, for example, U.S. Pat. No. 2,674,619, which is hereby incorporated by reference in its entirety, and Newman et al. (1998, Critical Reviews in Therapeutic Drug Carrier Systems 15(2): 89-142), which is also hereby incorporated by reference in its entirety.

The data presented herein shows that a small amount of plasmid DNA may weakly associate with a POE-POP-POE copolymer such as CRL-1005 alone. A central theme of the present invention is the generation of physically distinct particles as compared for example to CRL-1005, wherein all three components are associated, which further promotes the association of plasmid DNA to the block copolymer of the present invention and in turn results in a marked enhancement of a cellular immune response. Therefore, a central theme of the present invention is the disclosure that by combining a polynucleotide, a block copolymer and a cationic surfactant, it is possible to substantially increase association of the polynucleotide to the copolymer/cationic surfactant-based adjuvant, and in turn, effectively enhance the cellular immune response. Although the data in the Example sections suggest that the association of plasmid DNA to the CRL-1005 particles leads to an improved immune response, the mechanism by which the immune response is enhanced is at present unclear. While not being bound by theory in any way, it is possible that DNA associated to CRL-1005 particles may be more readily taken up and expressed by cells. It is also possible that the negative surface charge of the CRL-1005 particles, produced by the association of plasmid DNA to CRL-1005/BAK particles, may be important for enhancing the adjuvant properties of CRL-1005. The data presented in the Example sections do not distinguish between these two possible mechanisms of enhancing the immune response. The measurement of surface charge (zeta potential) and the amount of DNA associated with CRL-1005 particles in the Example sections are consistent with a model for the interaction of plasmid DNA/the block copolymer (CRL-1005) and the cationic surfactant (for example, BAK). The model suggests that BAK binding to particles of CRL-1005, through hydrophobic interactions, results in a reduction of the CRL-1005 particle size and in the formation of positively charged CRL-1005 particles. Binding of the polynucleotide (plasmid DNA encoding HIV gag) is believed to occur through electrostatic interactions between the positively charged headgroup of the cationic surfactant (BAK) and the DNA phosphate groups, while the hydrophobic tail of the cationic surfactant is embedded within the block copolymer (CRL-1005) particle. It is disclosed herein that by warming a solution to room temperature which comprises a polynucleotide, a block copolymer such as a higher molecular weight poloxamer, and a cationic surfactant such as BAK, a population of microparticles will form which possess a surface charge and diameter which suggest an increased association of the polynucleotide molecules with the adjuvant (i.e., poloxamer-based particle) as compared to a composition which lacks the cationic surfactant. This increased polynucleotide association (see Example sections 1-5, Table 1 and Table 3) is linked to an increase an in vivo immune response in rhesus monkeys inoculated with a polynucleotide/poloxamer/BAK-containing microparticle wherein the polynucleotide expresses HIV gag (see, e.g., Example sections 7, 12, 13 and 14).

As noted above, the polynucleotide vaccine exemplified herein is HIV gag. However, it will be known to the artisan of ordinary skill in the art that numerous polynucleotide vaccine constructs are contemplated for the adjuvanted polynucleotide vaccines and method of use described herein for use in a vertebrate host, such as a human or non-human mammal, and especially in human and veterinary applications, such as a DNA plasmid vector expressing hemagglutinin (HA), a surface glycoprotein of influenza A, the nucleoprotein of influenza A, the HBsAg surface antigen from hepatitis B, as well as additional constructs from HIV, including but not limited to pol, nef, env (such as gp120, gp41 or a complete gp 160 construct), tat, and rev. Therefore, it is evident that this specification gives excellent guidance to the skilled artisan to utilize the nucleic acid formulations of the present invention with an additional construction not expressly exemplified in the Example sections. Therefore, numerous other constructs representing different DNA constructs, modes of delivery, disease and antigen targets are envisioned for use in the vaccine formulations of the present invention. Examples of viral or bacterial challenges which may be amenable to either a prophylactic or therapeutic treatment include but are not limited to influenza, herpes simplex virus (HSV), human immunodeficiency virus (HIV), tuberculosis, human papilloma virus, hepatitis A, hepatitis B, and hepatitis C. It will also be within the scope of the present invention to provide prophylactic or, most likely, therapeutic treatment for non-infectious diseases, such as cancer, autoimmune disorders, and various allergies. Additionally, it will be within the purview of the skilled artisan to utilize the formulations of the present invention for any number of veterinary applications, including but not limited to rabies, distemper, foot and mouth disease, anthrax, bovine herpes simplex and bovine tuberculosis.

The present invention relates to adjuvanted polynucleotide vaccine formulations which comprise in part a non ionic block copolymer. While the invention contemplates use of any such block copolymer which promotes generation of a particle size and surface charge as described herein, a preferred non ionic block copolymer are polyoxyethylene (POE)/polyoxypropylene (POP) block copolymers, especially higher molecular weight POE-POP-POE block copolymers. As noted in the Background of the Invention, these compounds are described in U.S. Reissue Pat. No. 36,665, U.S. Pat. No. 5,567,859, U.S. Pat. No. 5,691,387, U.S. Pat. No. 5,696,298 and U.S. Pat. No. 5,990,241, and WO 96/04392, all of which are hereby incorporated by reference. Briefly, these nonionic block copolymers have the following general formula:
HO(C₂H₄O)ₐ(C₃H₆O)_{b}(C₂H₄O)ₐ H

wherein (b) represents a number such that the molecular weight of the hydrophobic POP portion (C₃H₆O) is up to approximately 20,000 daltons and wherein (a) represents a number such that the percentage of hydrophilic POE portion (C₂H₄O) is between approximately 1% and 40% by weight.

A preferred POE-POP-POE block copolymer that can be used as a vaccine adjuvant has the following formula:
HO(C₂H₄O)ₐ(C₃H₆O)_{b}(C₂H₄O)ₐ H

wherein (b) represents a number such that the molecular weight of the hydrophobe (C₃H₆O) is between approximately 9000 Daltons and 15,000 Daltons and (a) represents a number such that the percentage of hydrophile (C₂H₄O) is between approximately 3% and 35%.

Another preferred POE-POP-POE block copolymer that can be used as a vaccine adjuvant has the following formula:
HO(C₂H₄O)ₐ(C₃H₆O)_{b}(C₂H₄O)ₐ H

wherein (b) represents a number such that the molecular weight of the hydrophobe (C₃H₆O) is between approximately 9000 Daltons and 15,000 Daltons and (a) represents a number such that the percentage of hydrophile (C₂H₄O) is between approximately 3% and 10%.

A further preferred surface-active copolymer that can be used as a vaccine adjuvant has the following formula:
HO(C₂H₄O)ₐ(C₃H₆O)_{b}(C₂H₄O)ₐ H

wherein (b) represents a number such that the molecular weight of the hydrophobe (C₃H₆O) is approximately 9000 Daltons and (a) represents a number such that the percentage of hydrophile (C₂H₄O) is approximately 3%.

Another preferred surface-active copolymer that can be used as a vaccine adjuvant has the following formula:
HO(C₂H₄O)ₐ(C₃H₆O)_{b}(C₂H₄O)ₐ H

wherein (b) represents a number such that the molecular weight of the hydrophobe (C₃H₆O) is approximately 12000 Daltons and (a) represents a number such that the percentage of hydrophile (C₂H₄O) is approximately 5%, which represents the structure of CRL-1005, wherein (a) is about 7, ±1 and (b) is approximately 12,000 Daltons, with about 207 units, ±7.

The adjuvanted polynucleotide vaccine formulations of the present invention also comprise a cationic surfactant. It will be known to one of skill in the art that numerous cationic surfactants may be a candidate for use in these formulations. Therefore, the invention contemplates use of any cationic surfactant which, along with a block copolymer, promotes generation of a particle size and surface charge as described herein. Cationic surfactants which may be used include but are not limited to benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC) and cetyl trimethylammonium chloride (CTAC), primary amines, secondary amines, tertiary amines, including but not limited to N,N′,N′-polyoxyethylene(10)-N-tallow-1,3-diaminopropane, other quaternary amine salts, including but not limited to dodecyltrimethylammonium bromide, hexadecyltrimethylammonium bromide, mixed alkyl-trimethyl-ammonium bromide, benzyldimethyldodecylammonium chloride, benzyl-dimethylhexadecylammonium chloride, benzyltrmethylammonium methoxide, cetyldimethylethylammonium bromide, dimethyldioctadecyl ammonium bromide, methylbenzethonium chloride, decamethonium chloride, methyl mixed trialkyl ammonium chloride, methyl trioctylammonium chloride), N,N-dimethyl-N-[2(2-methyl-4-(1,1,3,3-tetramethylbutyl)-phenoxy]-ethoxy)ethyl]-benzenemethanaminium chloride (DEBDA), dialkyldimetylammonium salts, N-[1-(2,3-dioleyloxy)-propyl]-N,N,N,-trimethylammonium chloride, 1,2-diacyl-3-(trimethylammonio)propane (acyl group=dimyristoyl, dipalmitoyl, distearoyl dioleoyl), 1,2-diacyl-3-(dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl-3-succinyl-sn-glycerol choline ester, cholesteryl (4′-trimethylammonio)butanoate), N-alkyl pyridinium salts (e.g. cetylpyridinium bromide and cetylpyridinium chloride), N-alkylpiperidinium salts, dicationic bolaforn electrolytes (Cl₂Me₆;Cl₂Bu₆), dialkylglycetylphosphorylcholine, lysolecithin, L-α dioleoyl phosphatidylethanolamine), cholesterol hemisuccinate choline ester, lipopolyamines, including but not limited to dioctadecylamidoglycylspermine (DOGS), dipalmitoyl phosphatidylethanol-amidospermine (DPPES), lipopoly-L(orD)-lysine (LPLL, LPDL), poly(L (orD)-lysine conjugated to N-glutarylphosphatidylethanolamine, didodecyl glutamate ester with pendant amino group (Cl₂GluPhCₙN⁺), ditetradecyl glutamate ester with pendant amino group (Cl₄GluCₙN⁺), cationic derivatives of cholesterol, including but not limited to cholesteryl-3β-oxysuccinamidoethylenetrimethylammonium salt, cholesteryl-3,β-oxysuccinamidoethylenedimethylamine, cholesteryl-3,β-carboxyamidoethylenetrimethylammonium salt, cholesteryl-3β-carboxyamidoethylenedimethylamine, and 3β[N-(N′,N′-dimethylaminoetane-carbomoil] cholesterol).

In a preferred aspect of the present invention, the cationic surfactant is selected from the group consisting of benzalkonium chloride, benzethonium chloride, cetramide, cetylpyridinium chloride and cetyl trimethylammonium chloride. Benzalkonium chloride is available commercially and is known to exist as a mixture of alkylbenzyldimethylammonium chlorides of the general formula:
[C₆H₅C₂N(CH₃)₂R]Cl,
where R represents a mixture of alkyls, including all or some of the group beginning with n-C₈H₁₇ through n-C₁₆H₃₃. The average MW of BAK is 360 (see Handbook of Pharmaceutical Excipients, Ed. Wade and Weller, 1994, 2^{nd} Ed.at page 27-29). Benzethonium chloride is N,N-Dimethyl-N-[2-[2-[4-(1,1,3,3-tetramethylbutyl)phenoxy]ethoxy]ethyl]benzene-methanaminium chloride (C₂₇H₄₂ClNO₂), which has a molecular weight of 448.10 (Id. at page 30-31). Cetramide consists mainly of trimethyltetradecylammonium bromide (C₁₇H₃₈BrN),which may contain smaller amounts of dodecyltrimethylammonium bromide (C₁₅H₃₄BrN) and hexadecyltrimethylammonium bromide (C₁₉H₄₂BrN), and has a molecular weight of 336.40 (Id. at page 96-98).

The essence of the present invention is the generation (at temperatures above the cloud point of CRL-1005, or another representative block copolymer) of microparticles which comprise a block copolymer and cationic surfactant in contact with polynucleotide molecules. It is the formation of this copolymer/surfactant adjuvant particle during warming which promotes enhanced association of polynucleotide molecules to the particle and in turn which enhances the adjuvant properties of this microparticle. It is disclosed within the Example section that the cationic surfactants benzalkonium chloride (BAK), benzethonium chloride, cetylpyridinium chloride and cetyltrimethylammonium chloride significantly enhance the association of plasmid DNA to particles of a POE-POP-POE block copolymer, CRL-1005. Immunogenicity results in Rhesus monkeys (Example Section 7) show that the addition of a cationic surfactant such as BAK to formulations containing HIV-FLgag plasmid DNA and CRL-1005 enhances the cellular immune response to HIV-gag, compared to formulations without BAK. In formulations containing 5 mg/mL plasmid DNA, 7.5 mg/mL CRL-1005 and 0.75 mM BAK (formulation D121), approximately 3% of the plasmid DNA is associated with the adjuvant.

Therefore, central to the present invention is the ability to generate a microparticle with an enhanced association with a polynucleotide, such as plasmid DNA. As noted above, although the data in the Example sections suggest that the association of plasmid DNA to the CRL-1005 particles leads to an improved immune response, the mechanism by which the immune response is enhanced is unclear. It is possible that DNA associated to CRL-1005 particles may be more readily taken up and expressed by cells. It is also possible that the negative surface charge of the CRL-1005 particles, produced by the association of plasmid DNA to CRL-1005/BAK particles, may be important for enhancing the adjuvant properties of CRL-1005. The data presented in the Example sections do not distinguish between these two possible mechanisms of enhancing the immune response. In one embodiment of the present invention the formed particle is from about 100 nm to about 2000 nm in diameter. The non-ionic block copolymer particle in the presence of the cationic surfactant will have a positive surface charge whereas the polymer particle in the presence of cationic surfactant and DNA should have a surface charge significantly more negative than the polymer particle alone. The exemplified mircoparticles described in the Example sections (e.g., see Table 3) range from about 200-600 nm in diameter with a slightly positive zeta potential measurement in the presence of BAK but without addition of the polynucleotide (about 2.5 mV for CRL-1005 and 0.71 mM BAK) and a negative zeta potential when the polynucleotide (at 5 mg/ml) is present (about −46.6 mV for CRL-1005 and 0.71 mM BAK and 5 mg/ml plasmid DNA). While these values are instructive, they are by no way limiting. The quantitative measurements described in Table 3 are useful to show structural characteristics of adjuvant and polynucleotide vaccine formulations. However, as important is the qualitative notion that the addition of a cationic surfactant changes the configuration or structural integrity of the particle, which in turn increases the ability of the altered structure to better interact with polynucleotide molecules. This increased ability for polynucleotide molecules to associate with a copolymer/surfactant-based particle has been shown herein to be related to an enhancement of an in vivo cellular immune response as compared to inoculation with the DNA/poloxamer alone. Therefore, while ranges of surface charge and size measurements of various particles may be instructive, they are not necessarily limiting. Instead, these measurements are instructive when practicing the claimed invention because it allows the artisan to document that use of one type of block copolymer with one type of cationic surfactant results in a distinct mircoparticle, a microparticle ultimately characterized by an increased ability to associate with a specific population of polynucleotide molecules. For example, Table 3 shows that plasmid DNA alone has a surface charge of about −48.5 mV and a hydrodynamic diameter of about 66 nm, plasmid DNA with CRL-1005 results in an adjuvant material with a surface charge of about −5.1 mV and a diameter of about 1387 nm, and CRL-1005 and 0.71 mM BAK results in yet another structurally distinct particle with a surface charge of about 2.5 mV and a diameter of about 226 nm. Finally, mixing all three components and bringing the solution above the cloud point results in yet another distinct particle with a surface charge of about −46.6 mV and a diameter of about 674 nm. And, as noted above, it is shown herein that this DNA/poloxamer/cationic surfactant particle contains a larger amount of associated polynucleotide (approximately 3%), which is in turn shown to enhance the in vivo cellular immune response. Therefore, these data are instructive in showing that mixing of various components does in fact result in distinct particles with distinct surface charge characteristics, and that a DNA/poloxamer/cationic surfactant may result in increased association of the vaccine portion with the adjuvant portion such that the adjuvant activity is enhanced. These or similar measurements will then be instructive to the skilled artisan to determine an appropriate match of one specific block copolymer adjuvants with another specific cationic surfactant.

In view of the foregoing, the present invention relates in part to adjuvants and related polynucleotide vaccine formulations with adjuvants which comprise a block copolymer and a cationic surfactant. The inclusion of a cationic surfactant results in an increased percentage of polynucleotide that is physically associated with the block copolymer/cationic surfactant upon mixing, thus resulting in an enhanced in vivo immune response to polynucleotide vaccines and/or gene therapy-based transgenes. To this end, the present invention relates in part to an adjuvant for use in polynucleotide vaccine and gene therapy-based applications wherein the adjuvant has an enhanced propensity to associate with a the negatively charged polynucleotide vaccine component, resulting in a greater percentage of polynucleotide within the vaccine formulation being associated with the adjuvant prior to host administration.

The present invention also relates to polynucleotide vaccine adjuvants which comprise a block copolymer and a cationic surfactant, wherein the block copolymer is a non-ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐ H, wherein (b) represents a number such that the molecular weight of the hydrophobic POP portion (C₃H₆O) is up to approximately 20,000 daltons and wherein (a) represents a number such that the percentage of hydrophilic POE portion (C₂H₄O) is between approximately 1% and 40% by weight.

One embodiment of the present invention is an adjuvant which comprises a POE-POP-POE block copolymer and cationic surfactant, wherein the block copolymer has the formula HO(C₂H₄O)ₐ(C₃H₆O)_{b}(C₂H₄O)ₐ H, wherein (b) represents a number such that the molecular weight of the hydrophobe (C₃H₆O) is between approximately 9000 Daltons and 15,000 Daltons and (a) represents a number such that the percentage of hydrophile (C₂H₄O) is between approximately 3% and 35%, preferably at or below 10%.

Another embodiment of the present invention is an adjuvant which comprises a POE-POP-POE block copolymer and cationic surfactant, wherein the block copolymer has the formula HO(C₂H₄O)ₐ(C₃H₆O)_{b}(C₂H₄O)ₐ H, wherein (b) represents a number such that the molecular weight of the hydrophobe (C₃H₆O) is approximately 9000 Daltons and (a) represents a number such that the percentage of hydrophile (C₂H₄O) is between approximately 3% and 5%.

Yet another embodiment of the present invention is an adjuvant which comprises a POE-POP-POE block copolymer and cationic surfactant, wherein the block copolymer has the formula HO(C₂H₄O)ₐ(C₃H₆O)_{b}(C₂H₄O)ₐ H, wherein (b) represents a number such that the molecular weight of the hydrophobe (C₃H₆O) is approximately 12000 Daltons and (a) represents a number such that the percentage of hydrophile (C₂H₄O) is approximately 5%, such as CRL-1005, wherein (a) is about 7 units, ±1 and (b) is approximately 12,000 Daltons, with about 207 units, ±7.

The present invention relates further to an adjuvant which contains one or more of the block copolymers described above and herein, as well as a cationic surfactant, including but not limited to a cationic surfactant selected from the group consisting of benzalkonium chloride (BAK), benzethonium chloride, cetramide, cetylpyridinium chloride (CPC) and cetyl trimethylammonium chloride (CTAC). Other cationic surfactants known in the art, and especially cationic surfactants listed within this specification, may be substituted for one or more of the above-listed cationic surfactants.

A specific embodiment of the present invention relates to an adjuvant which comprises a POE-POP-POE block copolymer of formula HO(C₂H₄O)ₐ(C₃H₆O)_{b}(C₂H₄O)ₐH, wherein (b) represents a number such that the molecular weight of the hydrophobe (C₃H₆O) is approximately 9000 Daltons and (a) represents a number such that the percentage of hydrophile (C₂H₄O) is between approximately 3% and 5%, and a cationic surfactant which is especially amenable to human administration, such a cationic surfactant being selected from the group consisting of benzalkonium chloride (BAK), benzethonium chloride, cetramide, cetylpyridinium chloride (CPC) and cetyl trimethylammonium chloride (CTAC).

Another embodiment of the present invention is an adjuvant which comprises a POE-POP-POE block copolymer of formula HO(C₂H₄O)ₐ(C₃H₆O)_{b}(C₂H₄O)ₐ H, wherein (b) represents a number such that the molecular weight of the hydrophobe (C₃H₆O) is approximately 12000 Daltons and (a) represents a number such that the percentage of hydrophile (C₂H₄O) is approximately 5%, and a cationic surfactant which, as noted above, is especially amenable to human administration, such a cationic surfactant being selected from the group consisting of benzalkonium chloride (BAK), benzethonium chloride, cetramide, cetylpyridinium chloride (CPC) and cetyl trimethylammonium chloride (CTAC).

A specific embodiment of the present invention is an adjuvant which comprises a POE-POP-POE block copolymer, CRL-1005 and a cationic surfactant selected from the group consisting of benzalkonium chloride (BAK), benzethonium chloride, cetramide, cetylpyridinium chloride (CPC) and cetyl trimethylammonium chloride (CTAC).

Another specific embodiment of the present invention is an adjuvant which comprises a POE-POP-POE block copolymer, CRL-1005 and the cationic surfactant benzalkonium chloride (BAK).

In addition to the adjuvants described above and herein, the present invention also relates to polynucleotide vaccine formulations which comprise a polynucleotide component and adjuvant component, the adjuvant component comprising a block copolymer and a cationic surfactant. These formulations will contain a percentage of polynucleotide associated with the adjuvant and a percentage of free polynucleotide (i.e., polynucleotide which is not associated with the adjuvant in vitro). The formulations of the present invention show an increased proportion of polynucleotide associated with the adjuvant in vitro, which in turn results in a concomitant enhancement in in vivo immune responses.

To this end, the polynucleotide vaccine formulations of the present invention relate to polynucleotide vaccine formulations which comprise a polynucleotide component, block copolymer and a cationic surfactant, wherein the block copolymer is a non ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐ H, wherein (b) represents a number such that the molecular weight of the hydrophobic POP portion (C₃H₆O) is up to approximately 20,000 daltons and wherein (a) represents a number such that the percentage of hydrophilic POE portion (C₂H₄O) is between approximately 1% and 40% by weight.

One embodiment of the present invention is an adjuvanted polynucleotide formulation which comprises a polynucleotide component, a POE-POP-POE block copolymer and cationic surfactant, wherein the block copolymer has the formula HO(C₂H₄O)ₐ(C₃H₆O)_{b}(C₂H₄O)ₐ H, wherein (b) represents a number such that the molecular weight of the hydrophobe (C₃H₆O) is between approximately 9000 Daltons and 15,000 Daltons and (a) represents a number such that the percentage of hydrophile (C₂H₄O) is between approximately 3% and 35%, preferably at or below 10%.

Another embodiment of the present invention is an adjuvanted polynucleotide vaccine formulation which comprises a polynucleotide component, a POE-POP-POE block copolymer and cationic surfactant, wherein the block copolymer has the formula HO(C₂H₄O)ₐ(C₃H₆O)_{b}(C₂H₄O)ₐ H, wherein (b) represents a number such that the molecular weight of the hydrophobe (C₃H₆O) is approximately 9000 Daltons and (a) represents a number such that the percentage of hydrophile (C₂H₄O) is between approximately 3% and 5%.

Yet another embodiment of the present invention is an adjuvanted polynucleotide vaccine formulation which comprises a polynucleotide component, a POE-POP-POE block copolymer and a cationic surfactant, the block copolymer having the formula HO(C₂H₄O)ₐ(C₃H₆O)_{b}(C₂H₄O)ₐ H, wherein (b) represents a number such that the molecular weight of the hydrophobe (C₃H₆O) is approximately 12000 Daltons and (a) represents a number such that the percentage of hydrophile (C₂H₄O) is approximately 5%.

An additional embodiment of the present invention is an adjuvanted polynucleotide vaccine formulation which comprises a polynucleotide component, a POE-POP-POE block copolymer and a cationic surfactant, wherein the block copolymer is CRL-1005.

In regard to an adjuvant comprising a cationic surfactant, the present invention relates more specifically to a polynucleotide vaccine formulation which comprises a polynucleotide component, a POE-POP-POE block copolymer of formula HO(C₂H₄O)ₐ(C₃H₆O)_{b}(C₂H₄O)ₐ H, wherein (b) represents a number such that the molecular weight of the hydrophobe (C₃H₆O) is approximately 9000 Daltons and (a) represents a number such that the percentage of hydrophile (C₂H₄O) is between approximately 3% and 5%, and a cationic surfactant which is especially amenable to human administration, such a cationic surfactant being selected from the group consisting of benzalkonium chloride (BAK), benzethonium chloride (BEC), cetramide, cetylpyridinium chloride (CPC) and cetyl trimethylammonium chloride (CTAC).

Another embodiment of the present invention is a polynucleotide vaccine formulation which comprises a polynucleotide component, an adjuvant which comprises a POE-POP-POE block copolymer of formula HO(C₂H₄O)ₐ(C₃H₆O)_{b}(C₂H₄O)ₐ H, wherein (b) represents a number such that the molecular weight of the hydrophobe (C₃H₆O) is approximately 12000 Daltons and (a) represents a number such that the percentage of hydrophile (C₂H₄O) is approximately 5% (such as CRL-1005, described herein), and a cationic surfactant which, as noted above, is especially amenable to human administration, such a cationic surfactant being selected from the group consisting of benzalkonium chloride (BAK), benzethonium chloride, cetramide, cetylpyridinium chloride (CPC) and cetyl trimethylammonium chloride (CTAC).

A specific embodiment of the present invention is a polynucleotide vaccine formulation which comprises a polynucleotide component, an adjuvant which comprises a POE-POP-POE block copolymer, CRL-1005 and a cationic surfactant selected from the group consisting of benzalkonium chloride (BAK), benzethonium chloride, cetramide, cetylpyridinium chloride (CPC) and cetyl trimethylammonium chloride (CTAC).

Another specific embodiment of the present invention is a polynucleotide vaccine formulation which comprises a polynucleotide component, an adjuvant which comprises a POE-POP-POE block copolymer, CRL-1005 and the cationic surfactant benzalkonium chloride (BAK).

Upon review of this specification, the artisan will be able to mix and match various polycations, poloxamers cationic surfactants, excipients, as well as utilize various concentrations of these components. The artisan will be able to measure in vitro structural characteristics of the adjuvant or vaccine formulation, as shown herein, which may be instructive in preparing such components for in vivo administration. It is preferred that the concentration range of a respective nucleic acid be from about 0.5 mg/ml to about 7.5 mg/ml, the poloxamer be at a concentration of from about 1 to about 70 mg/ml and that the cationic surfactant(s) be present at a concentration from about 0.1 to 10 mM. An even more preferred range of components would be the nucleic acid be from about 1 mg/ml to about 7 mg/ml, with 5-6 mg/ml being especially preferred, the poloxamer at a concentration of from about 5 to about 50 mg/ml and that the cationic surfactant(s) be present at a concentration from about 0.2 to about 2 mM. Some preferred formulations are shown in Table 2, such as formulation #D118 (5 mg/mL DNA, 7.5 mg/mL CRL-1005, 0.45 mM BAK in PBS) and D121 (5 mg/mL DNA, 7.5 mg/mL CRL-1005, 0.75 mM BAK in PBS), as well as D118a, disclosed in Table 7.

The present invention also relates to an adjuvant which comprises a block copolymer, a cationic surfactant as described within this specification and a non-ionic surfactant, such as polysorbate-80, which may be a useful excipient to control particle aggregation in the presence of the antigen, such as a polynucleotide. Additional non-ionic surfactants are known in the art and may be used to practice this portion of the invention. These additional non-ionic surfactants include but are not limited to other polysorbates, n-Alkylphenyl polyoxyethylene ether, n-alkyl polyoxyethylene ethers (e.g., Tritons™), sorbitan esters (e.g., Spans™), polyglycol ether surfactants (Tergitol™), polyoxyethylenesorbitan (e.g., Tweens™), poly-oxyethylated glycol monoethers (e.g., Brij™, polyoxylethylene 9 lauryl ether, polyoxyethylene 10 ether, polyoxylethylene 10 tridecyl ether), lubrol, copolymers of ethylene oxide and propylene oxide (e.g. Pluronic™, Pluronic R™, Tetronic™, Pluradot™), alkyl aryl polyether alcohol (Tyloxapol™), perfluoroalkyl polyoxylated amides, N,N-bis[3-D-gluconamidopropyl]cholamide, decanoyl-N-methylglucamide, n-decyl β-D-glucopyranozide, n-decyl β-D-glucopyranozide, n-decyl β-D-maltopyanozide, n-dodecyl β-D-glucopyranozide, n-undecyl β-D-glucopyranozide, n-heptyl β-D-glucopyranozide, n-heptyl β-D-thioglucopyranozide, n-hexyl β-d-glucopyranozide, n-nonanoyl β-glucopyranozide 1-monooleyl-rac-glycerol, nonanoyl-N-methylglucamide, n-dodecyl β-D-maltoside, n-dodecyl β-D-maltoside, N,N bis[3-gluconamidepropyl]deoxycholamide, diethylene glycol monopentyl ether, digitonin, heptanoyl-N-methylglucamide, hepanoyl-N-methylglucamide, octanoyl-N-methylglucamide, n-octyl βD-glucopyranozide, n-octyl βD-glucopyranozide, n-octyl β-D-thiogalactopyranozide, n-octyl β-d-thioglucopyranozide. Therefore, the present invention relates to an adjuvant comprising a non-ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐ H, wherein (b) represents a number such that the molecular weight of the hydrophobic POP portion (C₃H₆O) is less than approximately 20,000 daltons and wherein (a) represents a number such that the percentage of hydrophilic POE portion (C₂H₄O) is between approximately 1% and 40% by weight; a cationic surfactant and a non-ionic surfactant, the non-ionic surfactant useful to inhibit particle aggregation in the presence of a population of polynucleotide molecules.

To this end, the present invention also relates to a polynucleotide vaccine formulation which first comprises a polynucleotide, an adjuvant component comprising a block copolymer and a cationic surfactant, as described within this specification, and secondly comprising a non-ionic surfactant, such as polysorbate-80 or other excipients, including but not limited to excipients known in the art such as glycerol or propylene glycol, or a non-ionic surfactant listed herein, which may be a useful excipient to control particle aggregation. Therefore, the present invention relates to a polynucleotide vaccine comprising a non-ionic block copolymer such as polyoxyethylene (POE)/polyoxypropylene (POP) block copolymers, especially higher molecular weight POE-POP-POE block copolymers which have the general formula HO(C₂H₄O)ₐ(C₃H₆O)_{b}(C₂H₄O)ₐH, wherein (b) represents a number such that the molecular weight of the hydrophobic POP portion (C₃H₆O) is less than approximately 20,000 daltons and wherein (a) represents a number such that the percentage of hydrophilic POE portion (C₂H₄O) is between approximately 1% and 40% by weight; a cationic surfactant and a non-ionic surfactant, the non-ionic surfactant or other excipients such as glycerol and propylene glycol, useful to inhibit particle aggregation.

As noted in the Summary of the Invention, the present invention thus relates to adjuvants, polynucleotide vaccine compositions/formulations, and their respective methods of use, which are useful in promoting an effective immune response upon administration within a target host, such as a human or non-human mammal. The term “adjuvant” as used herein is not limit to a specific mode of action as it relates to the disclosed adjuvant compositions and formulations of the present invention. As noted below, the term “adjuvant” as used herein may refer to any substance or combination of substances which nonspecifically enhances the immune response to an antigen, such as an antigen expressed from a delivered polynucleotide, such that expression of an antigen from the polynucleotide results in an increased in vivo immune response. Included in this definition are substances which may act as facilitators of in vivo cellular gene delivery, thereby increasing the amount of plasmid DNA delivered to cells that can express the intended antigen. Substances which may enhance delivery of plasmid DNA would include those which do not substantially interact with the plasmid DNA in the formulation and substances which do interact with the plasmid DNA, forming tightly bound or weakly bound complexes between the adjuvant and the plasmid DNA, either in vitro or in vivo.

In view of the adjuvants, adjuvanted polynucleotide formulations and related pharmaceutically acceptable products disclosed herein, the present invention also relates to methods of generating an enhanced immune response and/or expression of a respective gene therapy product in a vertebrate host by administering to the host the adjuvanted polynucleotide vaccine formulations of the present invention. The preferred host is a mammalian host, preferable a human or non-human mammal of commercial or domestic veterinary importance. These vaccine or gene therapy formulations are administered to the host by any means known in the art of DNA vaccines, such as enteral and parenteral routes. These routes of delivery include but are not limited to intramuscular injection, intraperitoneal injection, intravenous injection, inhalation or intranasal delivery, oral delivery, sublingual administration, subcutaneous administration, transdermal administration, transcutaneous administration, percutaneous administration or any form of particle bombardment. The preferred methods of delivery are intramuscular injection, subcutaneous, intranasal and oral based deliveries. An especially preferred method is intramuscular delivery. The adjuvanted polynucleotide formulations described herein are administered to the host by any means known in the art, such as enteral and parenteral routes. These routes of delivery include but are not limited to intramuscular injection, intraperitoneal injection, intravenous injection, inhalation or intranasal delivery, oral delivery, sublingual administration, subcutaneous administration, transdermal administration, transcutaneous administration, percutaneous administration or any form of particle bombardment, such as a biolostic device such as a “gene gun” or by any available needle-free injection device. The preferred methods of delivery of the formulations described herein are intramuscular injection, subcutaneous injection and needle-free injection. An especially preferred method is intramuscular delivery.

The compositions of the present invention may be administered to the host in any manner, strategy and/or combination available. For instance, the compositions of the present invention may be utilized in a regimen which may include a monovalent or multivalent composition, various combined modality applications, and/or a prime/boost regimen to as to optimize, for example in a gene vaccination scenario, antigen expression and a concomitant cellular-mediated and/or humoral immune response. Therefore, the present invention provides for methods of administration which may involve a prime/boost regime utilizing an adjuvanted plasmid DNA formulation and administration of a viral vector, such as an adenoviral vector for introduction into mammalian tissue (e.g., see data presented in Example Section 14). There may be a predetermined minimum amount of time separating the administrations. Therefore, the individual can be given a first dose of plasmid vaccine, and then a second dose of plasmid vaccine. Alternatively, the individual may be given a first dose of a recombinant viral vaccine, such as an adenovirus vaccine, and then a second dose of another viral vector vaccine. In other embodiments, the plasmid vaccine is administered first, followed after a time by administration of the adenovirus vaccine. Conversely, the adenovirus vaccine may be administered first, followed by administration of plasmid vaccine after a time. In these embodiments, an individual may be given multiple doses of the same adenovirus serotype in either viral vector or plasmid form, or the virus may be of differing serotypes. These administration regimes will pertain to all gene administration technologies, such as gene vaccination and gene therapy applications. Therefore, the adjuvanted plasmid DNA vaccines of this invention may be administered alone, or may be part of a prime and boost administration regimen. A mixed modality priming and booster inoculation scheme will result in an enhanced immune response, particularly is pre-existing anti-vector immune responses are present. This one aspect of this invention is a method of priming a subject with the plasmid vaccine by administering the plasmid vaccine at least one time, allowing a predetermined length of time to pass, and then boosting by administering the adenoviral vaccine. Multiple primings typically, 1-4, are usually employed, although more may be used. The length of time between priming and boost may typically vary from about four months to a year, but other time frames may be used.

It will be evident upon review of this specification that enabling guidance is given to the skilled artisan to utilize the adjuvants and adjuvanted polynucleotide formulations of the present invention with any additional polynucleotide construction not specifically exemplified within the Example sections. The exemplified polynucleotide expresses the p55 gag protein from HIV. However, there are countless other vaccine and gene therapy constructions, many known in the art at this time, which will be amenable to the disclosed adjuvants and adjuvanted polynucleotide formulations of the present invention. This methodology will be useful in either a prophylactic or therapeutic mode, depending on the disease(s) or disorder(s) being treated. Therefore, numerous other constructs representing different polynucleotide vaccines, modes of delivery, disease and antigen targets are envisioned for use in the vaccine formulations of the present invention. Examples of viral or bacterial challenges which may be amenable to either a prophylactic or therapeutic treatment include but are in no way limited to various antigen(s) associated with influenza, herpes simplex virus (HSV), human immunodeficiency virus (HIV), tuberculosis, human papilloma virus, hepatitis A, hepatitis B, hepatitis C, Epstein Barr virus, chicken pox, measles, rotavirus, respiratory syncytial virus, parainfluenza virus, Bordetella pertussis, E. coli, salmonella, tuberculosis. It will also be within the scope of the present invention to utilize the adjuvants and adjuvanted polynucleotide vaccines of the present invention to provide prophylactic or, most likely, therapeutic treatment for non-infectious diseases, such as cancer, autoimmune disorders, and various allergies. Additionally, it will be within the purview of the skilled artisan to utilize the adjuvants and polynucleotide formulations of the present invention for any number of veterinary applications, including but not limited to rabies, distemper, foot and mouth disease, anthrax, bovine herpes simplex and bovine tuberculosis. In other words, the adjuvants of the present invention may easily be applied to other, non-exemplified vaccine or gene therapy based constructs. The essence of the present invention is an improved adjuvant for use in formulating polynucleotide vaccines or gene therapy vehicles. Utilization of the present invention will most likely improve the respective immune response or expression of the targeted transgene within the host, as compared to administration which does not utilize the block copolymer/cationic surfactant based adjuvants of the present invention. It will be well within the purview of the skilled artisan to embrace the teachings herein to test for and measure improved efficacy of the respective polynucleotide vaccine or gene therapy construction.

In regard to prophylactic and/or therapeutic treatment of HIV-1 this virus is the etiological agent of acquired human immune deficiency syndrome (AIDS) and related disorders. HIV-1 is an RNA virus of the Retroviridae family and exhibits the 5′ LTR-gag-pol-env-LTR 3′ organization of all retroviruses. The integrated form of HIV-1, known as the provirus, is approximately 9.8 Kb in length. Each end of the viral genome contains flanking sequences known as long terminal repeats (LTRs). The HV genes encode at least nine proteins and are divided into three classes; the major structural proteins (Gag, Pol, and Env), the regulatory proteins (Tat and Rev); and the accessory proteins (Vpu, Vpr, Vif and Nef).

The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6.

The pol gene encodes proteins necessary for virus replication; a reverse transcriptase, a protease, integrase and RNAse H. These viral proteins are expressed as a Gag-Pol fusion protein, a 160 kDa precursor protein which is generated via a ribosomal frame shifting. The viral encoded protease proteolytically cleaves the Pol polypeptide away from the Gag-Pol fusion and further cleaves the Pol polypeptide to the mature proteins which provide protease (Pro, P10), reverse transcriptase (RT, P50), integrase (IN, p31) and RNAse H (RNAse, p15) activities.

The nef gene encodes an early accessory HIV protein (Nef) which has been shown to possess several activities such as down regulating CD4 expression, disturbing T-cell activation and stimulating HIV infectivity.

The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells.

The tat gene encodes a long form and a short form of the Tat protein, a RNA binding protein which is a transcriptional transactivator essential for HIV-1 replication.

The rev gene encodes the 13 kDa Rev protein, a RNA binding protein. The Rev protein binds to a region of the viral RNA termed the Rev response element (RRE). The Rev protein is promotes transfer of unspliced viral RNA from the nucleus to the cytoplasm. The Rev protein is required for HIV late gene expression and in turn, HIV replication.

Gp120 binds to the CD4/chemokine receptor present on the surface of helper T-lymphocytes, macrophages and other target cells in addition to other co-receptor molecules. X4 (macrophage tropic) virus show tropism for CD4/CXCR4 complexes while a R5 (T-cell line tropic) virus interacts with a CD4/CCR5 receptor complex. After gp120 binds to CD4, gp41 mediates the fusion event responsible for virus entry. The virus fuses with and enters the target cell, followed by reverse transcription of its single stranded RNA genome into the double-stranded DNA via a RNA dependent DNA polymerase. The viral DNA, known as provirus, enters the cell nucleus, where the viral DNA directs the production of new viral RNA within the nucleus, expression of early and late HIV viral proteins, and subsequently the production and cellular release of new virus particles. Recent advances in the ability to detect viral load within the host shows that the primary infection results in an extremely high generation and tissue distribution of the virus, followed by a steady state level of virus (albeit through a continual viral production and turnover during this phase), leading ultimately to another burst of virus load which leads to the onset of clinical AIDS. Productively infected cells have a half life of several days, whereas chronically or latently infected cells have a 3-week half life, followed by non-productively infected cells which have a long half life (over 100 days) but do not significantly contribute to day to day viral loads seen throughout the course of disease.

Destruction of CD4 helper T lymphocytes, which are critical to immune defense, is a major cause of the progressive immune dysfunction that is the hallmark of HIV infection. The loss of CD4 T-cells seriously impairs the body's ability to fight most invaders, but it has a particularly severe impact on the defenses against viruses, fungi, parasites and certain bacteria, including mycobacteria.

Effective treatment regimens for HIV-1 infected individuals have become available recently. However, these drugs will not have a significant impact on the disease in many parts of the world and they will have a minimal impact in halting the spread of infection within the human population. As is true of many other infectious diseases, a significant epidemiologic impact on the spread of HIV-1 infection will only occur subsequent to the development and introduction of an effective vaccine. There are a number of factors that have contributed to the lack of successful vaccine development to date. As noted above, it is now apparent that in a chronically infected person there exists constant virus production in spite of the presence of anti-HIV-1 humoral and cellular immune responses and destruction of virally infected cells. As in the case of other infectious diseases, the outcome of disease is the result of a balance between the kinetics and the magnitude of the immune response and the pathogen replicative rate and accessibility to the immune response. Pre-existing immunity may be more successful with an acute infection than an evolving immune response can be with an established infection. A second factor is the considerable genetic variability of the virus. Although anti-HIV-1 antibodies exist that can neutralize HIV-1 infectivity in cell culture, these antibodies are generally virus isolate-specific in their activity. It has proven impossible to define serological groupings of HIV-1 using traditional methods. Rather, the virus seems to define a serological “continuum” so that individual neutralizing antibody responses, at best, are effective against only a handful of viral variants. Given this latter observation, it would be useful to identify immunogens and related delivery technologies that are likely to elicit anti-HIV-1 cellular immune responses. It is known that in order to generate CTL responses antigen must be synthesized within or introduced into cells, subsequently processed into small peptides by the proteasome complex, and translocated into the endoplasmic reticulum/Golgi complex secretory pathway for eventual association with major histocompatibility complex (MHC) class I proteins. CD8⁺ T lymphocytes recognize antigen in association with class I MHC via the T cell receptor (TCR) and the CD8 cell surface protein. Activation of naive CD8⁺ T cells into activated effector or memory cells generally requires both TCR engagement of antigen as described above as well as engagement of costimulatory proteins. Optimal induction of CTL responses usually requires “help” in the form of cytokines from CD4⁺ T lymphocytes which recognize antigen associated with MHC class II molecules via TCR and CD4 engagement. The adjuvants and formulations of the present invention will help promote cellular immune responses within the target host agains HIV-1 or other disease or disorder by increasing expression of the targeted antigen within the host cell.

The dosage regimen utilizing the adjuvanted polynucleotide vaccine formulations described herein may be selected in accordance with a variety of factors including type, level of pre-existing immunity to expressed antigen, species, age, weight, sex and medical condition of the patient; the severity of the condition to be treated; the route of administration; the renal, hepatic and cardiovascular function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. To date, the optimal amount of plasmid DNA for a single inoculation of a human host is within the range of about 1 μg to about 10 mg of DNA, and preferably from about 100 μg to about 6 mg, and especially from about 1 mg to about 5 mg of DNA. It is of course central to the present invention that by increasing the immune response by generating a polynucleotide vaccine adjuvant as described herein, which may in turn reduce the amount of polynucleotide necessary to administer to the patient. To this end, the present invention also relates to a method of enhancing the immune response to a polynucleotide vaccine which comprises administration of an adjuvanted formulation described herein which contains at least a polynucleotide, preferably a DNA plasmid which expresses the transgene or antigen of interest, a polyoxamer as described herein and a cationic surfactant containing a quaternary ammonium compound which acts as a cationic surfactant.

The DNA vector vaccines of the present invention may be formulated in any pharmaceutically effective formulation for host administration. Any such formulation may be, for example, a saline solution such as phosphate buffered saline (PBS). It will be useful to utilize pharmaceutically acceptable formulations which also provide long-term stability of the DNA vector vaccines of the present invention. During storage as a pharmaceutical entity, DNA plasmid vaccines undergo a physiochemical change in which the supercoiled plasmid converts to the open circular and linear form. A variety of storage conditions (low pH, high temperature, low ionic strength) can accelerate this process. Therefore, the removal and/or chelation of trace metal ions (with succinic or malic acid, or with chelators containing multiple phosphate ligands, or with chelating agents such as EDTA) from the DNA plasmid solution, from the formulation buffers or from the vials and closures, stabilizes the DNA plasmid from this degradation pathway during storage. In addition, inclusion of non-reducing free radical scavengers, such as ethanol or glycerol, are useful to prevent damage of the DNA plasmid from free radical production that may still occur, even in apparently demetalated solutions. Furthermore, the buffer type, pH, salt concentration, light exposure, as well as the type of sterilization process used to prepare the vials, may be controlled in the formulation to optimize the stability of the DNA vaccine. Therefore, formulations that will provide the highest stability of the DNA vaccine will be one that includes a demetalated solution containing a buffer (phosphate or bicarbonate) with a pH in the range of 7-8, a salt (NaCl, KCl or LiCl) in the range of 100-200 mM, a metal ion chelator (e.g., EDTA, diethylenetriaminepenta-acetic acid (DTPA), malate, inositol hexaphosphate, tripolyphosphate or polyphosphoric acid), a non-reducing free radical scavenger (e.g. ethanol, glycerol, methionine or dimethyl sulfoxide) and the highest appropriate DNA concentration in a sterile glass vial, packaged to protect the highly purified, nuclease free DNA from light. A particularly preferred formulation which will enhance long term stability of the DNA vector vaccines of the present invention would comprise a Tris-HCl buffer at a pH from about 8.0 to about 9.0; ethanol or glycerol at about 0.5-3% w/v; EDTA or DTPA in a concentration range up to about 5 mM; and NaCl at a concentration from about 50 mM to about 500 mM. The use of such stabilized DNA vector vaccines and various alternatives to this preferred formulation range is described in detail in PCT International Application No. PCT/US97/06655, PCT International Publication No. WO 97/40839, which is hereby incorporated by reference.

The following examples are provided to further define the invention, without limiting the invention to the specifics of the examples.

### EXAMPLE SECTION

Materials and Methods—A pharmaceutical grade non-ionic block copolymer which comprises a portion of the adjuvants and formulations of the present invention may be synthesized by methods previously described, such as U.S. Pat. No. 5,567,859 and Re U.S. Pat. No. 36,665, which are both hereby incorporated by reference. In addition, Newman et al., (1998, Critical Reviews in Therapeutic Drug Carrier Systems 15(2): 89-142), describes synthesis of these pharmaceutical non-ionic block copolymers, which is also incorporated by reference. Newman et al (id), U.S. Pat. No. 5,567,859 and U.S. Pat. No. Re 36,665 provide methods of producing these higher molecular weight poloxamers while also purifying the product away from unwanted byproducts and impurities. Pharmaceutical grade cationic surfactants used herein are commercially available and are identified and described in detail in, for example, Handbook of Pharmaceutical Excipients, 2^{nd} Ed., Edited by A. Wade and P. J. Weller, 1994, American Pharmaceutical Association, Washington; The Pharmaceutical Press, London).

The non-ionic block copolymer CRL-1005 was obtained from CytRx Corporation, Norcross, Ga. Benzalkonium chloride (BAK), benzethonium chloride (BEC), cetyl pyridinium chloride (CPC) and cetyl trimethylammonium chloride were purchased from Spectrum Chemical. Polysorbate-80 was purchased from Sigma Chemical Co.

The hydrodynamic diameter (Dh) and the surface charge (Zeta potential) of CRL-1005 particles in solution were measured using a Zetasizer 3000, model DTS5300, made by Malvern Instruments. Prior to size measurements the formulations were diluted by 10-50-fold into PBS, pH 7.2. Zeta potential measurements were performed after dilution of the formulations by ˜50 fold into 20 mM Tris-acetate, pH 7.2.

To measure the amount of DNA associated to the CRL-1005 particles a 200 microliter sample of the formulation to be tested was added to the top of a 3.0 mL 2-13% sucrose gradient. After centrifugation for 1.5 hours at 80,000 RPM (348,000×g) sixteen 200 microliter fractions were removed, starting from the top of the gradient. The concentrations of DNA and BAK in each fraction were then determined by UV spectrophotometry. The percent DNA associated was then calculated by using the relationship shown below.
% DNA associated=(amount of DNA in fractions 1-6 divided by the amount of DNA in all fractions) multiplied by 100.

Preparation of CRL-1005 formulations containing DNA and BAK: The formulations described herein were prepared by adding pure polymer to a cold (<5° C.) solution of plasmid DNA in PBS using a positive displacement pipette. The solution was then vortexed to solubilize the polymer. After complete solubilization of the polymer a clear solution is obtained at temperatures below the cloud point of the polymer (˜6-7° C.). BAK was then added to the DNA/CRL-1005 solution in PBS, by slow addition of a dilute solution of BAK dissolved in PBS. Typically the concentration of the BAK solution that was added to the DNA/CRL-1005 solution was approximately 4 mM. Typically the initial DNA concentration was approximately 6 mg/mL before the addition of polymer and BAK. The final DNA concentration was 5 mg/mL in each formulation. After the addition of BAK the formulation was vortexed extensively, while the temperature was allowed to increase from ˜2° C. to above the cloud point. The formulation was then placed on ice to decrease the temperature below the cloud point. Then, the formulation was vortexed while the temperature was allowed to increase from ˜2° C. to above the cloud point. This process (cooling and mixing while the temperature was allowed to increase from ˜2° C. to above the cloud point) was repeated several times, until the particle size of the formulation was in the range of 200-500 nm, as measured by dynamic light scattering. The formulation was then stored on ice until the solution was clear, then placed in storage at −70° C. Before use, the formulations were removed from −70° C. storage and allowed to thaw at room temperature.

V1Jns-FLgag—The construction of V1Jns-Flgag (also described herein as V1Jns-gag), the exemplified, but in no way limiting polynucleotide shown herein, was disclosed in PCT International Application No. PCT/US98/02293, Publication No. WO 98/34640, filed Aug. 13, 1998. The open reading frame for an HIV-1 p55 gag, wherein codons have been optimized for expression in humans, is shown below and is presented as SEQ ID NO:27. The initiating methionine (ATG codon) is represented by nucleotides 10-12 and the “TAA” stop codon runs from nucleotides 1510-1512 of SEQ ID NO:27, encoding a representative HV-1 p55 gag antigen. The synthetic gene segments for increased gag gene expression were converted to sequences having identical translated sequences but with alternative codon usage as defined by R. Lathe in a research article from J. Molec. Biol. Vol. 183, pp. 1-12 (1985) entitled “Synthetic Oligonucleotide Probes Deduced from Amino Acid Sequence Data: Theoretical and Practical Considerations”. The methodology described below to increase of expression of HIV gag gene segments was based on our hypothesis that the known inability to express this gene efficiently in mammalian cells is a consequence of the overall transcript composition. Thus, using alternative codons encoding the same protein sequence may remove the constraints on expression of gag. The specific codon replacement method employed may be described as follows: (1) identify placement of codons for proper open reading frame; (2) compare wild type codon for observed frequency of use by human genes; (3) if codon is not the most commonly employed, replace it with an optimal codon for high expression in human cells; (4) repeat this procedure until the entire gene segment has been replaced; (5) inspect new gene sequence for undesired sequences generated by these codon replacements (e.g., “ATTTA” sequences, inadvertent creation of intron splice recognition sites, unwanted restriction enzyme sites, etc.) and substitute codons that eliminate these sequences; and, (6) assemble synthetic gene segments and test for improved expression.

These methods were used to create the following synthetic gene segments for HIV gag creating a gene comprised entirely of optimal codon usage for expression. While the above procedure provides a summary of our methodology for designing codon-optimized genes for DNA vaccines, it is understood by one skilled in the art that similar vaccine efficacy or increased expression of genes may be achieved by minor variations is the procedure or by minor variations in the sequence.

### EXAMPLE 1

Effect of Benzalkonium Chloride (BAK) and Benzethonium Chloride (BEC) on the Particle Size of CRL-1005

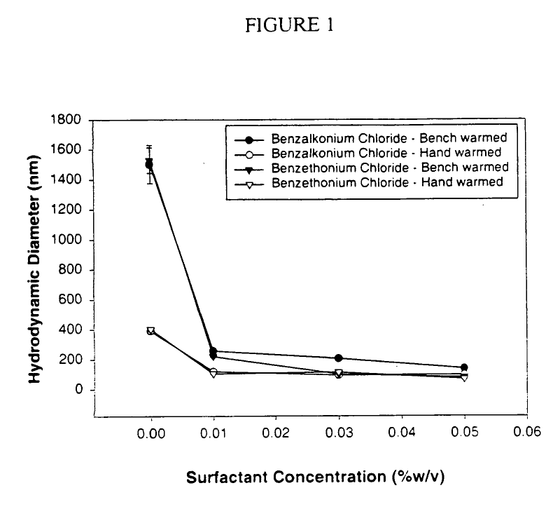

To determine the effect of BAK and BEC on the particle size of the POE-POP-POE block copolymer, CRL-1005, increasing amounts of BAK and BEC were added to formulations containing 7.5 mg/mL CRL-1005 in PBS. The samples were mixed well then frozen at −70° C. Before measuring the particle size, the samples were thawed by either placing the vial on the lab bench to thaw at room temperature or by warming the vial in the hand. The particle size of CRL-1005 was then determined by dynamic light scattering. The results in FIG. 1 indicate that BAK and BEC at concentrations as low as 0.01% reduced the particle size of CRL-1005 to ˜200 nm. The results also indicate that there was no significant effect of BAK or BEC concentration on particle size, between 0.01 and 0.05% surfactant. Moreover, the results indicate that there was very little effect of warming rate on particle size in the formulations containing BAK or BEC. These results suggest that both BAK and BEC stabilize a smaller particle size of CRL-1005 that is not significantly affected by the rate of warming through the cloud point.

### EXAMPLE 2

Effect of BAK on the Association of Plasmid DNA to CRL-1005 Particles

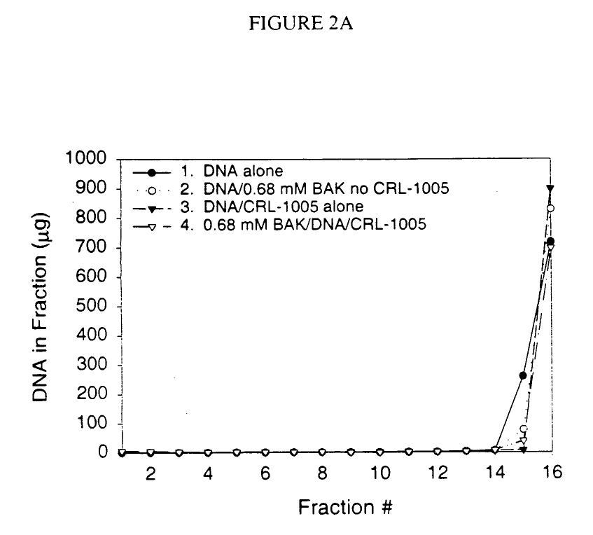

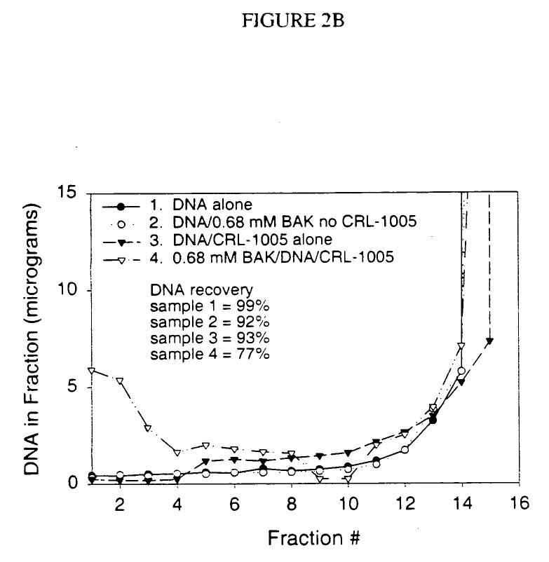

To determine whether BAK enhances the association of plasmid DNA to CRL-1005, an assay was devised to separate DNA associated to CRL-1005 from unassociated DNA. The method is based on the separation of associated and unassociated DNA using high speed centrifugation on a sucrose gradient. During centrifugation the plasmid DNA associated with CRL-1005 and BAK remains in the upper 1-6 fractions of the sucrose gradient while the majority of the unassociated DNA is pelleted. Using this assay a sample containing 7.5 mg/mL CRL-1005, 0.85 mM BAK and 5.0 mg/mL in PBS was placed on top of a 2-13% sucrose gradient and centrifuged at 80,000 rpm for 1.5 hours. The gradient was then fractionated into 16 fractions and the DNA concentration was determined in each fraction that contained polymer by direct UV absorbance measurements. The fractions containing polymer (1-6) were identified by visual observation. The results in FIGS. 2A and 2B show that the polymer containing fractions (1-6) also contain a significant amount of plasmid DNA, compared to the samples containing DNA alone, DNA+CRL-1005 or DNA+BAK. Since plasmid DNA was not retained in the upper fractions in the absence of CRL-1005, or in the absence of BAK, it is clear that both BAK and CRL-1005 are necessary to associate plasmid DNA to CRL-1005. For example, in sample 4, 2.5% of the total amount of DNA in all 16 fractions was located in the upper 6 fractions. For the sample containing CRL-1005 and DNA (sample 3), only ˜0.35% of the total DNA was found in the upper 6 fractions, suggesting that a very small amount of plasmid DNA was associated to CRL-1005 in the absence of BAK.

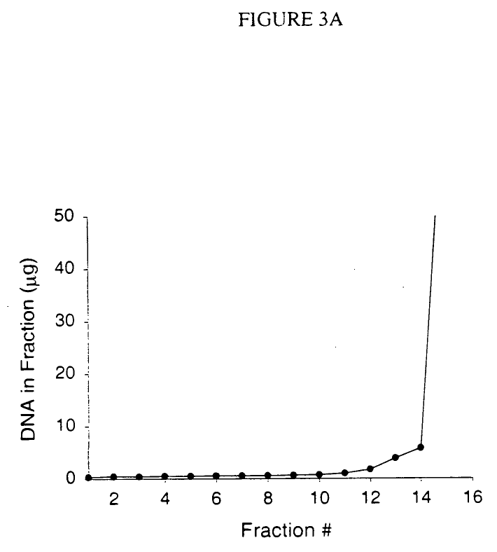

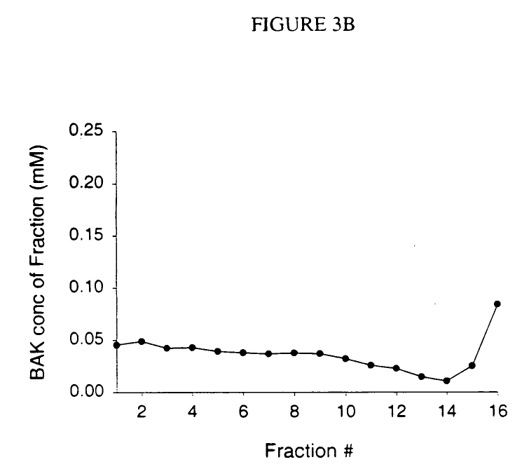

An analysis of the BAK concentration in each fraction of the gradient was performed for a sample containing only plasmid DNA and BAK. The results, shown in FIGS. 3A and 3B, indicate that BAK did not pellet with the free DNA (FIG. 3A) but was found almost evenly distributed throughout the gradient (see FIG. 3B). These results suggest that BAK does not bind to DNA in the absence of the CRL-1005 polymer or that the binding is weak and the BAK dissociates during the centrifugation run.

In a separate experiment a sample containing only BAK and CRL-1005 was centrifuged on the sucrose gradient followed by analysis of the fractions for BAK. The results showed that the BAK was found in fractions 1-6, co-localized with the polymer. Comparing these results to those of FIGS. 3A and 3B, it is clear that BAK binds to CRL-1005 particles more strongly than to plasmid DNA, in PBS.

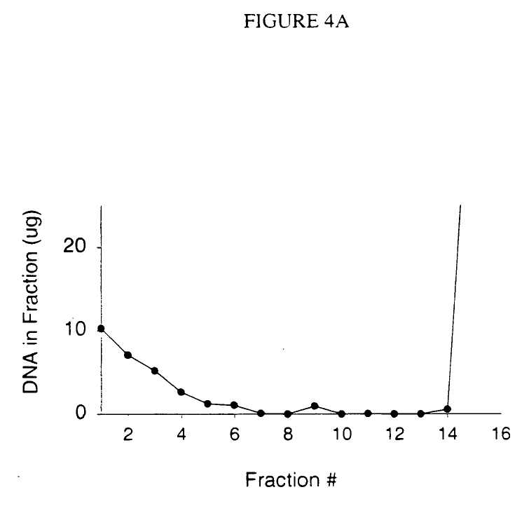

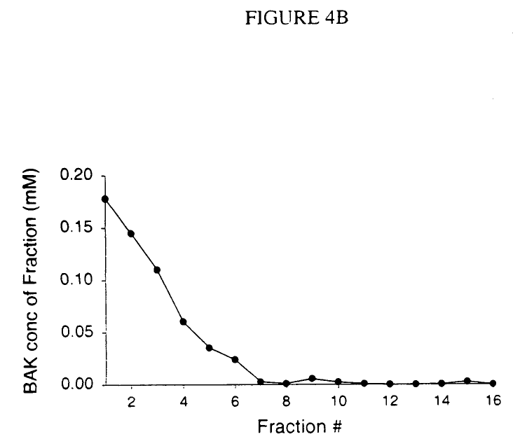

An analysis of the BAK concentration in each fraction of the gradient was also performed for a sample containing 5.0 mg/mL DNA/0.85 mM BAK/7.5 mg/mL CRL-1005. The results, shown in FIGS. 4A and 4B, indicate that the BAK (FIG. 4B) was found the upper six fractions of the gradient, co-localized with DNA (FIG. 4A) and with CRL-1005. The results also indicate that no BAK was detected in fractions 15-16 at the bottom of the tube with the unassociated DNA. Overall, these results suggest that BAK has a higher affinity CRL-1005 than for the DNA and that BAK mediates the interaction of plasmid DNA with CRL-1005.

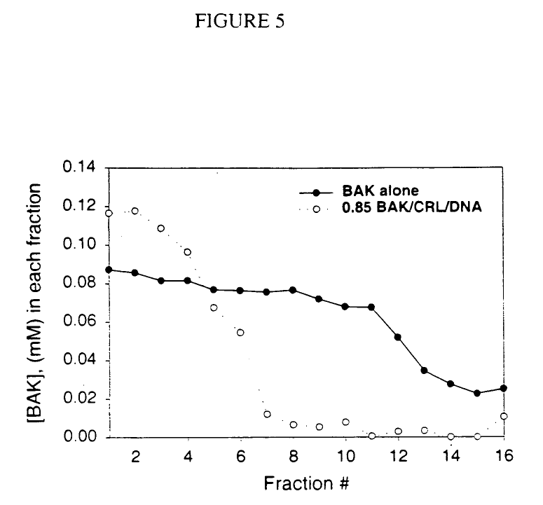

To determine whether BAK binds weakly to DNA a sample containing only BAK in PBS was centrifuged and the fractions assayed for BAK. A comparison of the BAK profile from this experiment (FIG. 5) to the results in FIGS. 3A-B and FIGS. 4A-B clearly shows that BAK binds weakly to DNA in the absence of CRL-1005.

The data in FIGS. 3A-B also indicate that a significant fraction of the BAK was found in fractions 8-16, while the data in FIG. 4A-B shows no significant BAK in fractions 8-16. These data indicate that free DNA-BAK complexes (not associated with CRL-1005 particles) were not present in the formulation, above the cloud point of CRL-1005. Since the D118 and D121 formulations are administered above the cloud point of CRL-1005 these data indicate that the enhanced immune response induced by formulation D118 was not due to the presence of free DNA-BAK complexes, but was due to CRL-1005 particles associated with BAK and DNA.

### EXAMPLE 3

Effect of BAK Concentration on the Association of Plasmid DNA to CRL-1005

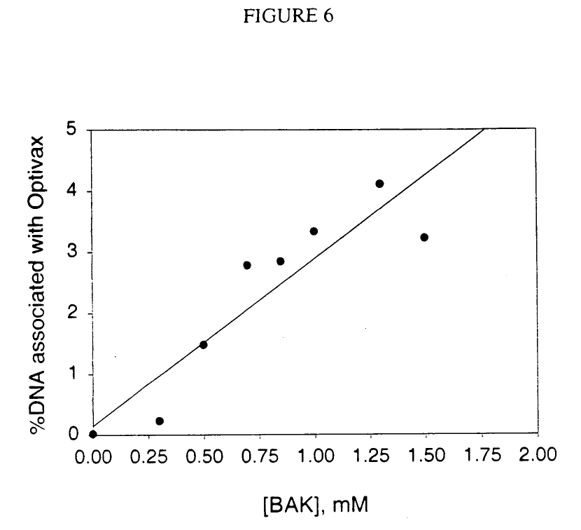

The benzalkonium chloride concentration was varied from 0 to 1.5 mM in a formulation containing 7.5 mM CRL-1005 to determine the effect of BAK concentration on the amount of plasmid DNA associated to the CRL-1005polymer. The results shown in FIG. 6 indicate that the amount of DNA association increases in a roughly linear fashion with BAK concentration, at least through 1.25 mM. The amount of DNA associated with CRL-1005 in formulation D121, which contains 0.75 mM BAK, 5 mg/mL DNA and 7.5 mg/mL CRL-1005, is typically 3.0-3.5%.

### EXAMPLE 4

Reproducibility & Stability of % Plasmid DNA Associated With CRL-1005

The amount of DNA associated with CRL-1005 was determined in a number of separate experiments using the sucrose gradient technique described above. The results are summarized in Table 1. The results indicate that the % DNA associated with CRL-1005 in formulation D121 (5 mg/mL DNA, 7.5 mg/mL CRL-1005, 0.85 mM BAK in PBS, pH 7.2) was ˜3.0-3.5%. However, in a few of the samples both lower and higher than expected values were obtained. For example, sample 12 had only 1.6% DNA associated after 24 hours at room temperature, suggesting that the % DNA association decreased over time. However, a repeat of this experiment (samples 15&16) indicated that the % DNA association was stable for 24 hours at room temperature. These data also indicate that the % DNA association was stable through at least four freeze/thaw cycles (see samples 17 & 18). The % DNA recovered was typically between 70 and 85% and the recovery did not appear to be correlated with the % DNA association.

TABLE 1
Percent DNA Associated to CRL-1005
Particles in Formulation D121

| Sample | % DNA | % DNA | Sample | |
|---|---|---|---|---|
| Number | Associated | Recovered | preparation | Comments |
| 1 | 3.3 | 78 | LM¹, in saline | |
| 2 | 4.3 | 67 | LM | |
| 3 | 5.4 | 91 | TWT², LM | |
| 4 | 6.8 | 87 | LM | |
| 5 | 3.0 | 64 | TWT, EM³ | |
| 6 | 2.8 | 89 | TWT, EM | |
| 7 | 2.9 | 83 | TWT, EM | |
| 8 | 3.0 | 78 | TWT, EM | |
| 9 | 2.2 | 84 | TWT, EM | |
| 10 | 2.7 | 79 | TWT, EM | 2 hours at 22 C. |
| 11 | 3.0 | 88 | TWT, EM | 6 hours at 22 C. |
| 12 | 1.6 | 98 | TWT, EM | 24 hours at 22 C. |
| 13 | 2.5 | 77 | TWT, EM | |
| 14 | 2.8 | 97 | TWT, EM | |
| 15 | 3.7 | 71 | TWT, EM | time zero sample |
| 16 | 3.5 | 73 | TWT, EM | incubated 24 hrs |
| | | | | at 22 C. |
| 17 | 3.5 | 86 | TWT, EM | one freeze/thaw |
| | | | | cycle |
| 18 | 3.6 | 84 | TWT, EM | four freeze/thaw |
| | | | | cycles |
| 19 | 3.4 | 72 | TWT, EM | |

¹Limited mixing.
²Tween washed centrifuge tubes.
³Extensive mixing.

### EXAMPLE 5

Effect of BAK Concentration on Heat Induced Aggregation

The effect of BAK concentration on heat induced aggregation was determined by determining the size of the CRL-1005 particles before and after one hour of incubation at 37° C. The formulations that were tested are described below in Table 2.

TABLE 2
Description of plasmid DNA formulations
containing CRL-1005 and BAK

| Formulation | [CRL-1005] | | Particle | |
|---|---|---|---|---|
| Code | mg/mL | Other Excipients | Size Range | |
| D118 | 7.5 | 0.45 mM BAK^{a} | 200-400 | nm |
| D120 | 7.5 | 0.45 mM BAK^{a} + 0.01% | ˜200 | nm |
| | | PS-80^{b} | | |
| D121 | 7.5 | 0.75 mM BAK^{a} | 300-500 | nm |
| D129 | 22.5 | 0.75 mM BAK^{a} | 300-500 | nm |
| D130 | 22.5 | 1.0 mM BAK^{a} | 300-500 | nm |
| D131 | 22.5 | 2.4 mM BAK^{a} | 300-500 | nm |

^{a}BAK = benzalkonium chloride
^{b}PS-80 = polysorbate-80

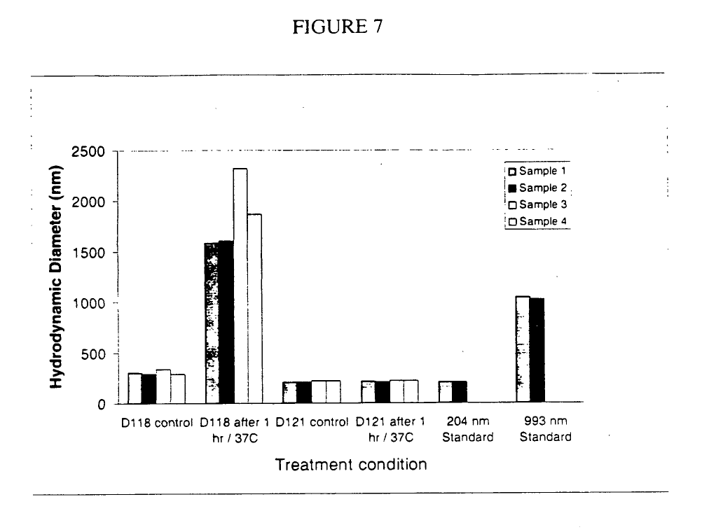

The results in FIG. 7 indicate that CRL-1005 particles in formulation D118 aggregate if incubated at 37° C. for one hour. However, the polymer particles did not aggregate under the same conditions in formulation D121, which contains a higher BAK concentration.

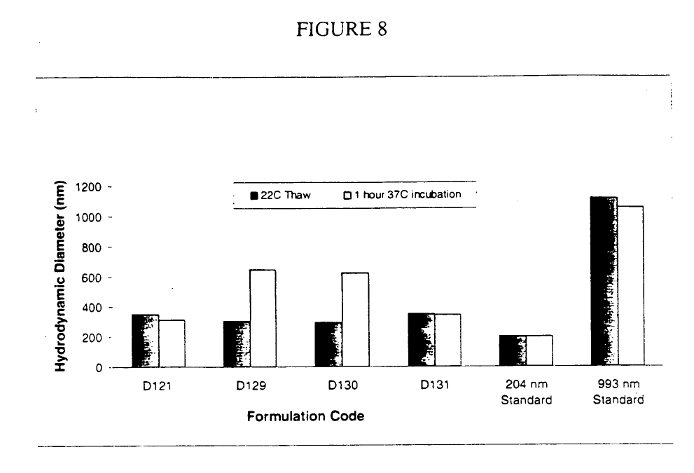

The effect of BAK concentration on the heat induced aggregation of CRL-1005 was also determined using formulations containing 22.5 mg/ML CRL-1005. The data in FIG. 8 indicate that 2.4 mM BAK is needed to prevent aggregation of CRL-1005 particles in formulations containing 22.5 mg/mL CRL-1005. These results indicate that a minimum ratio of BAK to CRL-1005 is needed to prevent aggregation at 37° C.

The amount of DNA associated with CRL-1005 particles appears to be stable for at least 24 hours at room temperature and was not affected by multiple freeze/thaw cycles. The particle size of CRL-1005 in formulation D121 was typically in the range of 300-500 nm, and was reproducible from preparation to preparation. A minimum ratio of BAK to CRL-1005polymer was necessary to eliminate aggregation after heating for one hour at 37° C.

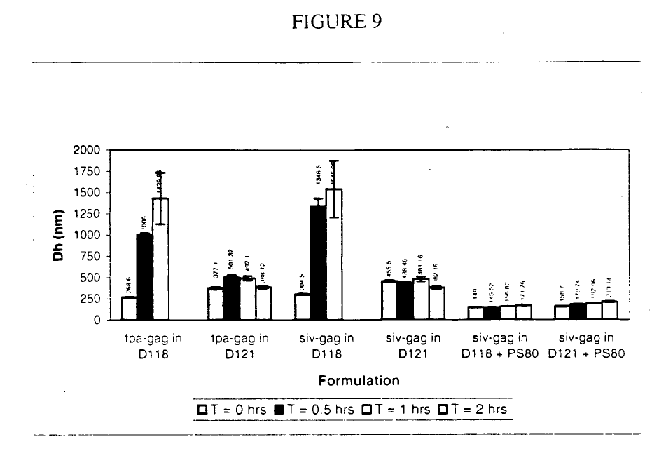

To determine if the addition of polysorbate-80 to formulation D118 would inhibit the aggregation of CRL-1005, 0.01% polysorbate-80 was added to D118 formulations containing either tpa-gag plasmid DNA or siv-gag plasmid DNA. The formulations were frozen, thawed at 30° C. and then held at 30° C. for up to 2 hours. Particle size measurements were performed by dynamic light scattering at time zero and after 0.5, 1 and 2 hours of incubation. The results, shown in FIG. 9, indicate that the addition of 0.01% polysorbate-80 prevented the aggregation of CRL-1005 in formulation D118, containing either form of plasmid DNA. Moreover, the control formulations (D121) did not aggregate in the presence or absence of polysorbate-80. These data indicate that polysorbate-80 effectively controls the aggregation of CRL-1005 in formulations containing BAK.

### EXAMPLE 6

Effect of BAK and Plasmid DNA on the Surface Charge of CRL-1005 Particles.

To determine the effect of BAK and DNA on the surface charge of CRL-1005 particles, zeta potential measurements were performed on several formulations. The concentrations of CRL-1005 and DNA used were 7.5 mg/mL and 5.0 mg/mL, respectively. The results, shown in Table 3, indicate that in the absence of BAK and DNA, particles of CRL-1005 are ˜1500 nm in size and have a zeta potential of −4.4 mV. DNA alone has a zeta potential of −48.5 mV, with a size of ˜70 nm. For the formulation containing DNA and CRL-1005, the zeta potential of the CRL-1005 particles is −5.1 mV, slightly more negative than the polymer in the absence of DNA. Although the difference in the zeta potential between CRL-1005 particles in the presence and absence of DNA is small, this difference appears to be consistent and suggests that a small amount of plasmid DNA is associated with particles of CRL-1005, in the absence of BAK. The data in FIG. 2 are also consistent with this hypothesis.

The zeta potential of CRL-1005 particles in the presence of 0.71 mM BAK (no DNA) was 2.5 mV. This result suggests that BAK binds to the CRL-1005 particles, changing the zeta potential of the particles from slightly negative to positive When plasmid DNA was present in the formulation containing CRL-1005 and BAK, the zeta potential changed from positive to highly negative. These data shows that binding of plasmid DNA to CRL-1005 particles containing BAK results in the formation of a negatively charged CRL-1005 particle containing both BAK and DNA.

To determine the effect of polysorbate-80 (PS-80) on the binding of plasmid DNA to CRL-1005 particles, the zeta potential of CRL-1005 particles in the presence of 0.71 mM BAK and 0.01% PS-80 was first determined and found to be ˜4.9 mV. When plasmid DNA was included in the formulation containing CRL-1005, BAK and 0.01% PS-80, the zeta potential changed from positive (in the absence of DNA) to very negative (in the presence of DNA). These results suggest that PS-80 did not greatly inhibit the binding of plasmid DNA to the CRL-1005 particles. However, the zeta potential of the CRL-1005 particles in the presence of DNA, BAK and 0.01% PS-80 is slightly less negative than the same formulation in the absence of PS-80, suggesting that there may be slightly less DNA bound in the presence of PS-80. The size of the CRL-1005 particles in the formulation containing DNA, CRL-1005, 0.71 mM BAK and 0.01% PS-80 was also smaller than the same formulations without PS-80, suggesting that PS-80 inhibits the aggregation of CRL-1005 particles when DNA is bound.

TABLE 3
Zeta Potential Measurements of CRL-1005 Formulations

| | Zeta potential | Dh |
|---|---|---|
| Formulation | (mV) | (nm) |
| CRL-1005 only | −4.4 | 1532 |
| DNA only | −48.5 | 66 |
| DNA + CRL-1005 | −5.1 | 1387 |
| CRL-1005 + 0.71 mM BAK | 2.5 | 226 |
| DNA + CRL-1005 + 0.71 mM | −46.6 | 674 |
| BAK | | |
| CRL-1005 + 0.71 mM | 4.9 | 220 |
| BAK + 0.01% PS-80 | | |
| PS-80 added before BAK | | |
| DNA + CRL-1005 + 0.71 mM | −41.3 | 305 |
| BAK + 0.01% PS-80 | | |
| PS-80 added before BAK | | |
| −50 +/− 5 mV zeta | −49.2, −48.9, −48.2, −46 | Not |
| potential standard | | applicable |
| 204 +/− 6 nm size standard | Not applicable | 202 |
| 993 +/− 21 nm size standard | Not applicable | 998 |

### EXAMPLE 7

Immunogenicity of Plasmid DNA/CRL-1005 Formulations in Rhesus Macaques

Experiment Design—Cohorts of 6 monkeys were vaccinated with the following:(1) 5 mg V1Jns-gag DNA in PBS; (2) 5 mg V1Jns-gag+7.5 mg CRL-1005; (3) 5 mg V1Jns-gag+22.5 mg CRL-1005, and cohorts of three monkeys were vaccinated with (4) 5 mg V1Jns-gag+22.5 mg CRL-1005+0.01% polysorbate-80 (PS-80), and (5) with 5 mg V1Jns-gag+7.5 mg CRL-1005+0.5 mM benzalkonium chloride. All vaccines were stored frozen in glass vials and a plastic secondary container; just prior to injection, the vials were slowly warmed to room temperature. Immunizations were conducted at 0 and 4 weeks. Each animal was vaccinated with 0.5 mL of vaccine in each deltoid, using an IM route of administration.

Results—Peripheral blood mononuclear cells (PBMCs) from the vaccinees were assayed at several time points via elispot assay and at 4 wks post-dose 2 via bulk CTL killing assay. The overall frequencies of antigen-specific T cells were marginally improved (50%) by formulating the DNA with 7.5 mg CRL-1005 (see Table 4A below, groups 1 & 2). However, overall frequencies of antigen-specific T cells were enhanced ˜2-fold by formulating the DNA with 22.5 mg/mL CRL-1005 (comparing groups 1 & 3). Moreover, greater than 4-fold increases in IFN-gamma secreting gag-specific T cells were observed in 2 of 3 monkeys by mixing CRL-1005 with the cationic detergent, BAK (comparing groups 1 & 5 in Tables 4A and 4B, respectively). This poloxamer/detergent/DNA formulation also led to an increased CD8+ component to the overall T cell response and a concomitant enhancement in the bulk CTL killing activity, relative to the poloxamer/DNA or DNA control groups.

TABLE 4A
Evaluation of Plasmid DNA with Various CRL-1005 Formulations
on HIV-1 gag Immunogencity in Rhesus Monkeys
Number of SFC per million PBMCs.

| Group^{a}/ | | | | | | | | | |
|---|---|---|---|---|---|---|---|---|---|
| Adjuvant | ID | Pre-bleed | | T = 4 weeks | | T = 6 weeks | | T = 8 weeks | |
| formulatio) | number | media | Gag H | media | Gag H | media | Gag H | media | Gag H |
| 1 | W253 | 16 | 28 | 19 | 53 | 11 | 74 | 9 | 49 |
| no adjuvant | W253(CD4-) | | | | | 3 | 4 | | |
| (D101) | W256 | 6 | 9 | 14 | 63 | 15 | 90 | 6 | 166 |
| | W256(CD4-) | | | | | 8 | 23 | | |
| | W257 | 23 | 43 | 6 | 83 | 14 | 388 | 6 | 140 |
| | W257(CD4-) | | | | | 15 | 106 | | |
| | 163H | 9 | 11 | 8 | 96 | 10 | 155 | 3 | 166 |
| | 163H(CD4-) | | | | | 11 | 35 | | |
| | 172H | 11 | 16 | 9 | 69 | 14 | 71 | 8 | 120 |
| | 172H(CD4-) | | | | | 10 | 35 | | |
| | 97002 | 11 | 10 | 13 | 83 | 16 | 86 | 6 | 53 |
| | 97002(CD4-) | | | | | 9 | 20 | | |
| 2 | 60H | 9 | 20 | 11 | 89 | 13 | 199 | 11 | 106 |
| 7.5 mg | 60H(CD4-) | | | | | 18 | 18 | | |
| CRL1005 | 60G | 8 | 5 | 14 | 135 | 10 | 225 | 3 | 99 |
| (D113) | 60G(CD4-) | | | | | 6 | 9 | | |
| | W274 | 6 | 6 | 6 | 91 | 9 | 174 | 5 | 39 |
| | W274(CD4-) | | | | | 8 | 14 | | |
| | T324 | 8 | 3 | 8 | 208 | 16 | 130 | 6 | 118 |
| | T324(CD4-) | | | | | 11 | 20 | | |
| | 83H | 6 | 6 | 6 | 70 | 20 | 68 | 10 | 144 |
| | 83H(CD4-) | | | | | 16 | 20 | | |
| | 106H | 10 | 13 | 15 | 104 | 20 | 278 | 15 | 195 |
| | 106H(CD4-) | | | | | 35 | 49 | | |
| 3 | 98C073 | 10 | 13 | 5 | 159 | 5 | 290 | 1 | 81 |
| 22.5 mg | 98C073(CD4-) | | | | | 4 | 29 | | |
| CRL1005 | | 13 | 10 | 9 | 130 | 5 | 164 | 5 | 309 |
| (D117) | 124H | | | | | 10 | 13 | | |
| | 124H(CD4-) | 30 | 8 | 9 | 129 | 11 | 155 | 6 | 173 |
| | 135H | | | | | 9 | 55 | | |
| | 135H(CD4-) | 13 | 13 | 8 | 150 | 8 | 345 | 8 | 486 |
| | 96078 | | | | | 8 | 111 | | |
| | 96078(CD4-) | 3 | 9 | 15 | 178 | 8 | 270 | 9 | 165 |
| | W270 | | | | | 6 | 34 | | |
| | W270(CD4-) | 8 | 4 | 8 | 109 | 3 | 68 | 5 | 100 |
| | 41G | | | | | 9 | 36 | | |
| | 41G(CD4-) | | | | | | | | |

^{a}Groups 1-3 received 5 mg or FL-gag plasmid DNA formulated in PBS either with or without adjuvant.

TABLE 4B
Evaluation of Plasmid DNA with Various CRL-1005 Formulations
on HIV-1 gag Immunogencity in Rhesus Monkeys
Number of SFC per million PBMCs.

| Group^{a}/ | | | | | | | | | |
|---|---|---|---|---|---|---|---|---|---|
| Adjuvant | ID | Pre-bleed | | T = 4 weeks | | T = 6 weeks | | T = 8 weeks | |
| (formulation) | number | media | Gag H | media | Gag H | media | Gag H | media | Gag H |
| 4 | 137Q | 2 | 8 | 21 | 159 | 5 | 153 | 14 | 99 |
| 22.5 mg | 137Q(CD4-) | | | | | 9 | 66 | | |
| CRL1005 + | S206 | 5 | 5 | 18 | 348 | 11 | 815 | 3 | 495 |
| 0.01% PS-80 | S206(CD4-) | | | | | 6 | 19 | | |
| (D119) | T331 | 6 | 6 | 13 | 70 | 8 | 105 | 6 | 109 |
| | T331(CD4-) | | | | | 9 | 4 | | |
| 5 | T319 | 0 | 3 | 18 | 76 | 16 | 139 | 11 | 144 |
| 7.5 mg | T319(CD4-) | | | | | 6 | 14 | | |
| CRL1005 + | 98C045 | 6 | 3 | 30 | 438 | 10 | 711 | 10 | 619 |
| 0.45 mM | 98C045(CD4-) | | | | | 24 | 163 | | |
| BAK | W258 | 2 | 5 | 15 | 421 | 6 | 538 | 5 | 326 |
| (D118) | W258(CD4-) | | | | | 24 | 183 | | |
| 6 | 040F | 5 | 10 | 25 | 23 | 20 | 19 | 3 | 5 |
| Naïve | 050F | 6 | 15 | 28 | 21 | NA | NA | 13 | 10 |
| controls | | | | | | | | | |

^{a}Groups 4-5 received 5 mg of FL-gag plasmid DNA formulated in PBS either with or without adjuvant.

### EXAMPLE 8

Enhancement of DNA Association to CRL-1005 Using Other Cationic Surfactants.

Two other cationic surfactants, cetylpyridinium chloride (CPC) and cetyl trimethylammonium chloride (CTAC) were formulated with 5 mg/mL DNA and 7.5 mg/mL CRL-1005. After freezing and thawing the sample the % DNA associated with the CRL-1005polymer was determined. The results indicate 4.7% association for CPC and 4.8% association for CTAC. In a previous experiment, 3.2% DNA association using benzethonium chloride was observed in the same formulation. These results clearly indicate that other cationic surfactants can enhance the association of DNA to the CRL-1005 particles.

An effective vaccine must induce an appropriate response to the correct antigen or antigens. There are several distinct types of immune responses which vary in their ability to confer protection against particular diseases. For example, antibodies may confer protection against bacterial infections, but cell mediated immunity is required for eliminating from the body many viral infections and tumors. There are multiple distinct types of antibody and cell-mediated immune responses. Cell-mediated responses are divided into tow basic groups: 1) delayed-type hypersensitivity in which T cells act as helper or suppressor cells indirectly via macrophages and other cells or cell products and via indirect interactions through products secreted from the T Cells such as cytokines, and 2) cytotoxicity in which specialized T-cells specifically and directly attack and kill infected cells.

### EXAMPLE 9

BAK Binds to CRL1005 Particles.

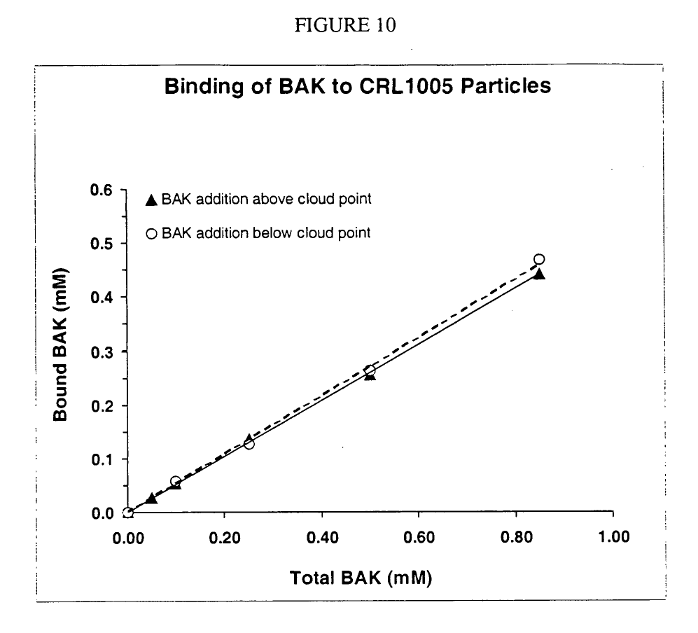

The data presented in Examples 2 and 6 shows that BAK binds to CRL-1005 particles. To determine the extent of binding, BAK was mixed with 7.5 mg/mL ICRL-1005 at temperatures above and below the cloud point. The mixtures were then incubated at room temperature for 30-60 minutes and centrifuged at 440,000×g at 25° C. for 30 minutes, to separate BAK bound to CRL-1005 particles from free BAK in solution. The sedimented material was resuspended in 2% SDS (to dissolve CRL-1005) and the BAK concentrations were measured by UV spectroscopy. The results indicated that ˜50% of the BAK was adsorbed to the CRL-1005 particles in the concentration range of BAK shown in FIG. 10. These data also indicate that the time of BAK addition did not affect the amount of BAK adsorbed. These results clearly indicate that BAK binds to CRL-1005 particles and supports the hypothesis that binding of plasmid DNA to CRL-1005 particles is mediated by BAK.

The BAK used in the present studies is a mixture of BAK homologs with chain lengths of 12, 14 and 16 carbons. An analysis of the BAK used for these experiments, by reverse phase HPLC, indicated that the stock BAK consisted of 68% BAK-12, 24% BAK14 and 8% BAK-16. Since the binding of BAK to CRL-1005 may depend on the chain length of BAK, we determined the fraction of BAK-12, BAK-14 and BAK-16 bound to CRL-1005 particles. For this study two formulations were prepared, each of which contained 7.5 mg/mL CRL-1005 and 5.0 mg/mL plasmid DNA in PBS. One of the formulations contained 0.45 mM BAK and the second formulation contained 0.85 mM BAK. The formulations were prepared and centrifuged as described above to sediment the BAK bound to the CRL-1005 particles. The pellets were redissolved with 2% SDS and the amount of BAK present was quantitated by UV spectroscopy. The amount of BAK-12, BAK-14 and BAK-16 in the supernatant was determined by reverse phase HPLC. The results, shown in Table 5, indicated that all of the BAK-14 and BAK-16 was bound to the CRL-1005 particles, while only about 18-25% of the BAK-12 was bound. These results show that BAK binds to CRL-1005 particles and suggest that the binding is stronger for BAK-14 and BAK-16 than for BAK-12. These results also indicate that plasmid DNA did not inhibit the binding of BAK to CRL-1005 particles.

TABLE 5
Fraction of BAK Bound to CRL1005 Particles
in PBS above the Cloud Point

| Formulation | | | |
|---|---|---|---|
| (all contain 7.5 mg/mL CRL1005) | BAK-12 | BAK-14 | BAK-16 |
| +0.45 mM BAK | 19% | 100% | 100% |
| +0.45 mM BAK + 5 mg/mL | 18% | 100% | 100% |
| plasmid DNA | | | |
| +0.75 mM BAK | 20% | 100% | 100% |
| +0.75 mM BAK + 5.0 mg/mL | 25% | 100% | 100% |
| plasmid DNA | | | |

### EXAMPLE 10

Comparing DNA/BAK Formulations with and without CRL-1005.

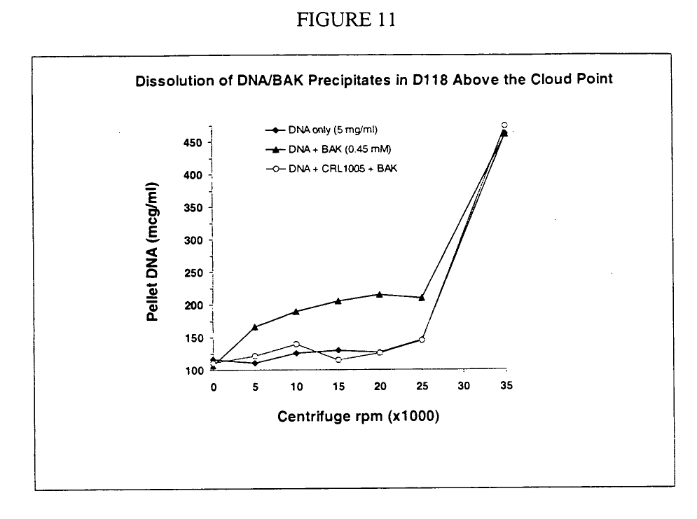

It was previously reported, in WO 99/21591, that BAK enhances the immune response to an antigen encoded in plasmid DNA. Moreover, it was claimed that the enhancement of immune response was due to the presence of soluble ionic complexes formed by plasmid DNA and BAK. The data presented in Example 9 indicate that BAK binds to CRL-1005 particles, but that most of the BAK-12 was free and not adsorbed to the CRL-1005 particles. These results suggest that free BAK-12 may potentially be bound to plasmid DNA and that DNA/BAK complexes (not associated with CRL-1005) may have been responsible for the enhancement in cellular immune response induced by DNA/BAK/CRL-1005 formulations. However, the data presented in Example 2 suggest that free DNA/BAK complexes do not exist in formulations containing DNA/BAK/CRL-1005, above the cloud point of CRL-1005. To further confirm these results, 0.4 mL samples of a formulation containing 7.5 mM CRL-1005, 5.0 mg/mL plasmid DNA and 0.45 mM BAK (D118) were centrifuged above the cloud point at several different speeds (from 5,000 to 40,000 rpm) for 30 minutes at 25° C. The range of centrifugation speeds was selected to be high enough to pellet BAK/DNA complexes but not high enough to pellet DNA associated with the CRL-1005/BAK particles. The pellets from each of the samples was dissolved in 0.4 ml of 2% SDS in PBS, and the DNA in the pellets was quantitated using UV spectroscopy. The results, from the step centrifugation of formulation D118 as well as two different controls are shown in FIG. 11.

The results indicate that for the DNA control (5.0 mg/mL DNA in PBS), 30 minutes of centrifugation at speeds <25,000 rpm did not pellet the DNA; the basal level of DNA recovered from the centrifuge tubes was due to residual amounts of DNA remaining in the tube after the sample was removed. In contrast, for a control sample containing 5.0 mg/mL DNA and 0.45 mM BAK, a significant amount (3.3%) of the DNA was pelleted at the lowest centrifugation speed (5,000 rpm). The amount of pelleted DNA reached a plateau (about 4% of the total DNA) at 15,000 rpm, clearly indicating the existence of DNA/BAK precipitates in this sample. In the D118 formulation, the amount of DNA pelleted was very similar to that in the DNA control. The absence of significant amounts of DNA in the pellet from D118 clearly indicates that DNA/BAK complexes were not present in this formulation.

These data indicate that the DNA/BAK precipitates in D118, formed at temperatures below the cloud point, were completely dissolved after the formation of the CRL-1005 particles. We reason this event by the following mechanism: the adsorption of BAK to CRL-1005 particles reduces the free BAK concentration to a level below its critical micellar concentration (cmc) and consequently causes the dissociation of BAK micelles, which results in the dissolution of the BAK micelle-mediated DNA precipitates. Therefore, the means by which DNA/BAK/CRL-1005 formulations enhance the immune response does not require the presence of DNA/BAK complexes.

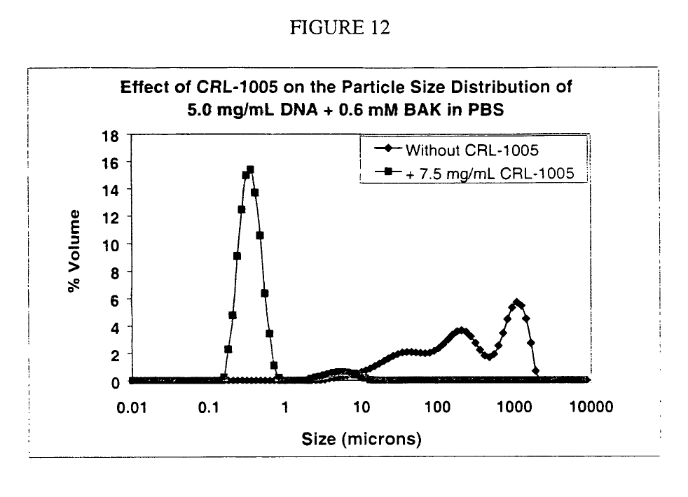

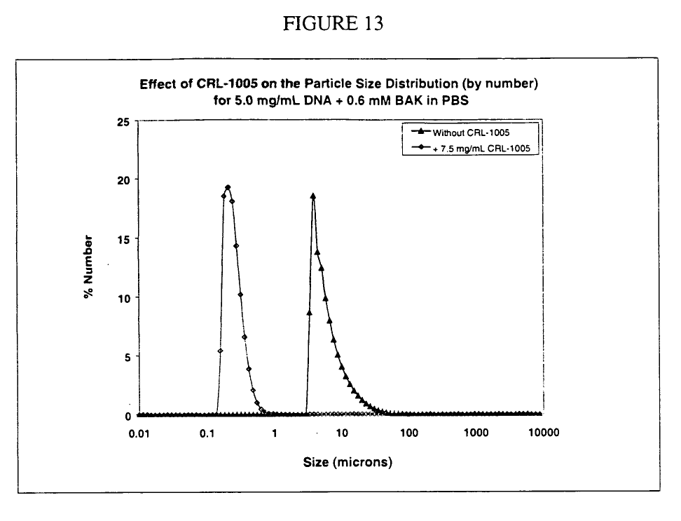

To further examine the effect of CRL-1005 on formulations containing DNA and BAK, static laser diffraction light scattering was used to determine the size distribution of DNA/BAK particles in an undiluted formulation containing 5.0 mg/mL DNA and 0.6 mM BAK in PBS. The results in FIG. 12 show a very broad particle size distribution in which a significant fraction of the particle volume (or mass) was present in particles with a size ranging from ˜100 to 2000 microns. In contrast, the particle size distribution (by volume) for D118a shows a much narrower distribution with a mean size near 300 nm. If the particle size distributions of D118a and the DNA/BAK formulation are shown plotted by number the results clearly indicate that the most numerous particles in the DNA/BAK mixture have particle sizes near 4 microns (see FIG. 13). In contrast, the analysis of particles in D118a shows that the distribution by number significantly overlaps the distribution by volume. These results clearly indicate that CRL-1005 significantly decreases the mean particle size and size distribution of formulations containing DNA and BAK.

### EXAMPLE 11

Effect of BAK on the Particle Size Distribution of DNA/CRL-1005 Formulations.

The particle size distributions of several DNA/CRL-1005 formulations used in animal studies (Table 6) were determined by static laser diffraction light scattering.

TABLE 6
Selected Plasmid DNA Formulations Tested in Rhesus Macques.

| Formulation | [DNA] | [CRL-1005] | | |
|---|---|---|---|---|
| Code | mg/mL | mg/mL | Buffer | Other Excipients |
| D113 | 5.0 | 7.5 | PBS | None |
| D118 | 5.0 | 7.5 | PBS | 0.45 mM BAK |
| D118a | 5.0 | 7.5 | PBS | 0.60 mM BAK |
| D145 | 5.0 | 7.5 | PBS | 0.70 mM BAK-12 |

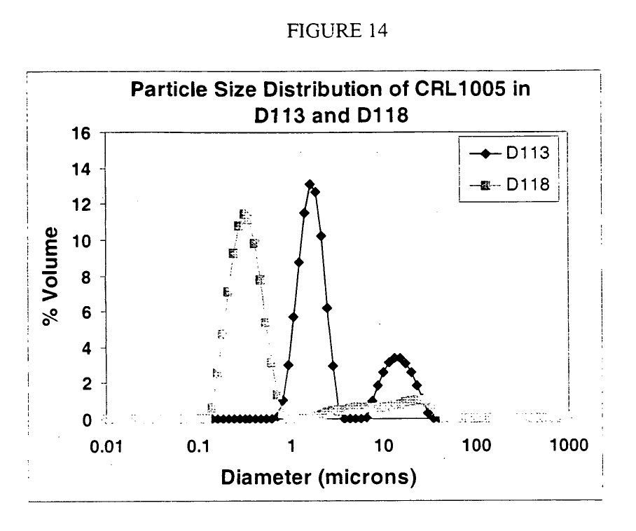

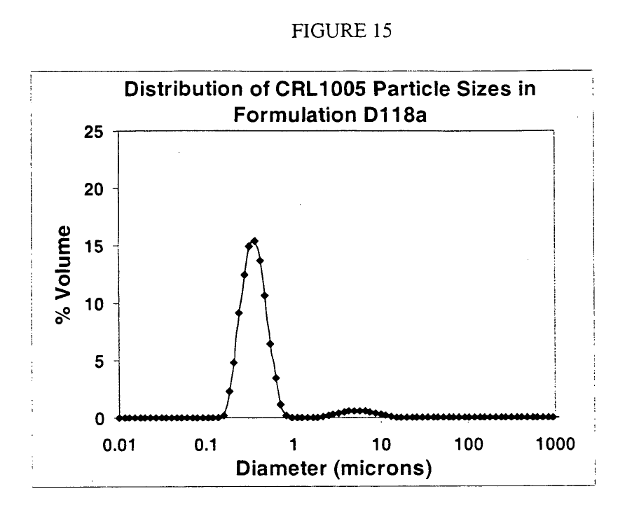

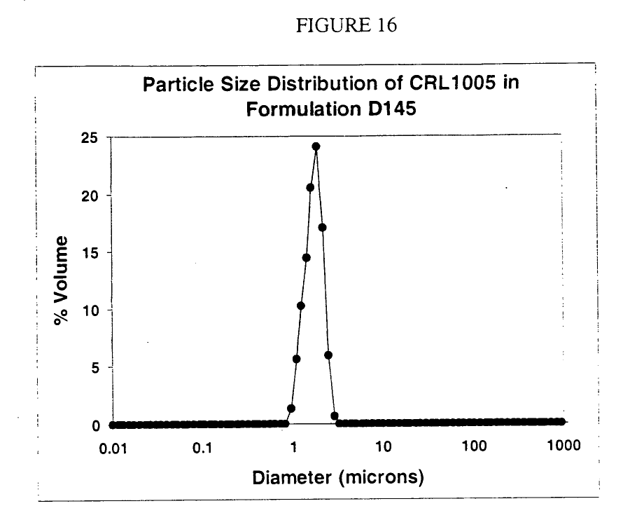

The data in FIG. 14 shows that BAK has a significant effect on the particle size distribution of CRL-1005, decreasing the mean particle size from ˜1500 nm to ˜300 nm. BAK also appears to reduce the amount of large (>10 micron) particles. Comparing the results in FIG. 14 with those in FIG. 12 indicates that when BAK is added to a formulation containing DNA in PBS large (˜1000 micron) particles are formed, but when BAK is added to a formulation containing DNA/CRL-1005 in PBS much smaller (˜300 nm) particles are formed. Therefore, BAK has very different effects on the particle size distribution of plasmid DNA formulations, depending on whether CRL-1005 is present or not. FIG. 15 shows the particle size distribution of D118a, which is similar to D118, but has a higher BAK concentration and contains a BAK with a significantly different chain composition than the BAK used in D118. The results indicate that the particle size distribution of D118a is not significantly different from that of D118, indicating that the changes in the BAK composition had little effect on the particle size distribution. The analysis of D145 (FIG. 16) indicates that the use of BAK-12 produced particles with a narrow size distribution in the 1-2 micron size range.

### EXAMPLE 12

Immunogenicity of Plasmid DNA/CRL1005/BAK Formulations in Rhesus Macaques.

Experiment Design—Cohorts of 3-5 monkeys were vaccinated with the following:(1) 5 mg V1Jns-gag DNA in PBS (D101); (2) 5 mg V1Jns-gag in D118; (3) 5 mg V1Jns-gag in D118a; (4) 5 mg V1Jns-gag in D145. The adjuvants vary with respect to the concentration of BAK used (0.45 vs 0.6 mM) and the type of BAK (BAK mixture vs single-chain length BAK-12). All vaccines were stored frozen in glass vials and a plastic secondary container; just prior to injection, the vials were slowly warmed to room temperature. Immunizations were conducted at 0, 4, 8 weeks. Each animal was vaccinated with 0.5 mL of vaccine in each deltoid, using an IM route of administration. Peripheral blood mononuclear cells (PBMCs) from the vaccinees were assayed at several time points via elispot assay against HIV-1 gag peptide pool (Table 7).

TABLE 7
Effect of BAK Type and Composition on Immunogenicity of
Plasmid DNA/CRL1005 Formulations in Rhesus Macaques.
Number of SFC per million PBMCs.

| | Vaccination | | T = 0 | | T = 4 | | T = 6 | | T = 10 | |
|---|---|---|---|---|---|---|---|---|---|---|
| Group# | (Formulation Code) | Monkey# | Medium | gag H | Medium | gag H | Medium | gag H | Medium | gag H |
| 1 | DNA in PBS | CB5H | ND | ND | 3 | 35 | 15 | 71 | 4 | 224 |
| | (D101) | CC6X | 0 | 0 | 0 | 15 | 0 | 46 | 0 | 58 |
| | | AW3G | 5 | 11 | 0 | 36 | 3 | 51 | 3 | 46 |
| 2 | DNA + | CC1X | 0 | 0 | 1 | 60 | 3 | 200 | 3 | 445 |
| | CRL-1005/7.5 mgs + | AW3W | 5 | 5 | 1 | 15 | 1 | 70 | 0 | 169 |
| | 0.45 mM BAK | AV43 | 2 | 1 | 3 | 20 | 3 | 110 | 0 | 241 |
| | (D118) | | | | | | | | | |
| 3 | DNA + | AW20 | 10 | 4 | 1 | 59 | 5 | 264 | 19 | 425 |
| | CRL-1005/7.5 mgs + | CA4R | 1 | 0 | 3 | 121 | 1 | 135 | 1 | 270 |
| | 0.6 mM BAK | CB58 | 8 | 6 | 0 | 6 | 3 | 119 | 0 | 274 |
| | (D118a) | CB5W | 4 | 3 | 0 | 26 | 1 | 91 | 0 | 139 |
| | | CB7D | 1 | 0 | 0 | 136 | 0 | 316 | 1 | 609 |
| 4 | DNA + | CC8K | 3 | 0 | 0 | 348 | 3 | 201 | 3 | 901 |
| | CRL-1005/7.5 mgs + | CC21 | 0 | 0 | 3 | 31 | 0 | 53 | 0 | 171 |
| | 0.7 mM BAK-12 | AW38 | 1 | 4 | 0 | 101 | 1 | 208 | 4 | 446 |
| | (D145) | 98D209 | 0 | 0 | 0 | 15 | 0 | 70 | 0 | 311 |
| 5 | none | 98D201 | 3 | 0 | 0 | 0 | 1 | 0 | 0 | 0 |

At wk 10, the overall frequencies of antigen-specific T cells were 2-4 fold higher when the DNA was formulated with any of the CRL-1005/BAK adjuvants. These results indicate DNA/BAK/CRL-1005 formulations prepared with varying concentrations of BAK (from 0.45 mM to 0.6 mM) or prepared with BAK of a single chain length (BAK-12) were able to enhance the immune response above that induced by plasmid DNA in PBS.

All of the formulations contained 5.0 mg/mL HIV-FLgag plasmid DNA and 7.5 mg/mL CRL-1005 in PBS. The BAK used in formulation D118 consisted of 68% BAK-12, 24% BAK-14 and 8% BAK-16 and was obtained from Spectrum Chemical. The BAK used in formulations D118a and D121 (described in Example 13 below) consisted of 50% BAK-12, 30% BAK-14, 17% BAK-16 and 3% BAK-18 and was obtained from Ruger Chemical Co.

A comparison of the immunogenicity results of Table 7 with the particle size distribution data in FIGS. 14-16 suggests that CRL-1005 particles in the size range from ˜300 nm to ˜2 microns were equally effective for enhancing the immune response.

### EXAMPLE 13

Immunogenicity of Plasmid DNA Formulations Containing CRL-1005 and/or BAK.

Experiment Design—Cohorts of 4 monkeys were vaccinated with the following:(1) 5 mg V1Jns-gag DNA in PBS (D101); (2) 5 mg V1Jns-gag+7.5 mg CRL-1005 (D113); (3) 5 mg V1Jns-gag+7.5 mg CRL-1005+0.45 mM BAK (D118); (4) 5 mg V1Jns-gag+7.5 mg CRL-1005+0.6 mM BAK (D121) and (5) 5 mg V1Jns-gag+0.45 mM BAK (D149). All vaccines were stored frozen in glass vials and a plastic secondary container; just prior to injection, the vials were slowly warmed to room temperature. Immunizations were conducted at 0, 4, and 8 weeks. Each animal was vaccinated with 0.5 mL of vaccine in each deltoid, using an IM route of administration. Peripheral blood mononuclear cells (PBMCs) from the vaccinees were assayed at several time points via elispot assay against HIV-1 gag peptide pool (gag H, Table 8A).

TABLE 8A
Effect of BAK on the Immunogenicity of Plasmid DNA/CRL1005 Formulations in Rhesus Macaques.
Number of SFC per million PBMCs.

| | Vaccination | | T = 0 | | T = 4 | | T = 6 | | T = 12 | |
|---|---|---|---|---|---|---|---|---|---|---|
| Group# | (Formulation Code) | Monkey# | Medium | gag H | Medium | gag H | Medium | gag H | Medium | gag H |
| 1 | HIV-FL-gag DNA + | 98C023 | 14 | 10 | 6 | 114 | 18 | 548 | 0 | 380 |
| | CRL-1005/7.5 mgs + | 73H | 14 | 21 | 0 | 486 | 0 | 671 | 0 | 1405 |
| | 0.45 mM BAK | 152H | 9 | 11 | 4 | 9 | 3 | 114 | 5 | 58 |
| | (D118) | W208 | 1 | 1 | 1 | 55 | 6 | 158 | 0 | 161 |
| 2 | HIV-FL-gag DNA + | W276 | 18 | 5 | 1 | 10 | 1 | 195 | 0 | 140 |
| | CRL-1005/7.5 mgs + | 98C043 | 9 | 10 | 3 | 58 | 5 | 433 | 0 | 560 |
| | 0.75 mM BAK | W285 | 9 | 8 | 1 | 35 | 3 | 329 | 0 | 266 |
| | (D121) | 98C004 | 0 | 3 | 0 | 25 | 5 | 111 | 0 | 333 |
| 3 | HIV-FL-gag DNA + | W289 | 10 | 28 | 0 | 19 | 5 | 121 | 1 | 183 |
| | CRL-1005/7.5 mgs | 98C002 | 9 | 13 | 0 | 39 | 1 | 169 | 0 | 283 |
| | (D113) | 98C019 | 23 | 19 | 0 | 13 | 0 | 219 | 1 | 123 |
| | | 98C068 | 0 | 4 | 4 | 11 | 3 | 128 | 4 | 220 |
| 4 | HIV-FL-gag DNA + | 98C086 | 0 | 0 | 0 | 6 | 1 | 164 | 1 | 291 |
| | 0.45 mM BAK | 98C104 | 0 | 1 | 5 | 16 | 0 | 244 | 1 | 461 |
| | (D149) | 98C152 | 0 | 0 | 3 | 24 | 1 | 100 | 0 | 253 |
| | | 98C155 | 1 | 0 | 0 | 16 | 1 | 299 | 4 | 465 |
| 5 | HIV-FL-gag DNA | 98C156 | 0 | 0 | 3 | 8 | 0 | 33 | 0 | 139 |
| | (D101) | 98D071 | 0 | 1 | 1 | 10 | 4 | 85 | 1 | 8 |
| | | 98D072 | 1 | 4 | 1 | 11 | 1 | 83 | 0 | 96 |
| | | 98D077 | 0 | 1 | 1 | 31 | 1 | 83 | 3 | 74 |
| 6 | none | 98D201 | 1 | 3 | 1 | 0 | 1 | 1 | 3 | 0 |
| | | 98D209 | 0 | 0 | 0 | 0 | 0 | 1 | 0 | 0 |

At wk 12, the overall frequencies of gag-specific T cells were 2-5 fold improved when comparing to DNA alone (D101) with any of the DNA/adjuvant formulations. The responses to the D118 formulation was about 2-fold better than the responses to D113 and the enhanced response with D118 was observed after only one injection (see data for wk 4). The responses to DNA in D118 were marginally higher than the responses to DNA in D149 at wk 12. However, the data also show that the immune response induced by DNA in D118 was observed after only one injection (see data for wk 4), while the response to DNA in D149 was not observed until after two injections (see data for wk 6). Moreover, the immune response induced by DNA in D118 was higher than that for DNA in D149 after two injections.

In a second experiment cohorts of 3 monkeys were vaccinated with the following:(1); 5 mg SIV gag DNA with D121 (7.5 mg CRL1005+0.75 mM BAK) and (2); 5 mg SIV gag DNA with 0.75 mM BAK (D150). All vaccines were stored frozen in glass vials and a plastic secondary container; just prior to injection, the vials were slowly warmed to room temperature. Immunizations were conducted at 0, 4, and 8 weeks. Each animal was vaccinated with 0.5 mL of vaccine in each deltoid, using an IM route of administration. Peripheral blood mononuclear cells (PBMCs) from the vaccinees were assayed at several time points via elispot assay against a Mamu-A01*-specific CD8⁺ T cell peptide epitope, p11c (Table 8B).

TABLE 8B
Effect of BAK on the Immunogenicity of Plasmid DNA/CRL1005 Formulations.
Number of SFC per trillion PBMCs.

| | | | T = 0 | | T = 4 | | T = 6 | | T = 15 | |
|---|---|---|---|---|---|---|---|---|---|---|
| | | | | SIV | | SIV | | SIV | | SIV |
| | Vaccination | | | gag | | gag | | gag | | gag |
| Group# | (Formulation Code) | Monkey# | Medium | p11c | Medium | p11c | Medium | p11c | Medium | p11c |
| 1 | SIV gag DNA + | 98D273 | 0 | 1 | 0 | 5 | 1 | 43 | 1 | 21 |
| | CRL1005/7.5 mg + | 98D318 | 0 | 0 | 1 | 8 | 1 | 959 | 0* | 1310 |
| | 0.75 mM BAK | 98D359 | 0 | 0 | 0 | 225 | 9 | 831 | 1* | 1630 |
| | (D121) | | | | | | | | | |
| 2 | SIV gag DNA + | 98X005 | 0 | 0 | 0 | 125 | 1 | 341 | 0 | 140 |
| | 0.75 mM BAK | 98D320 | 0 | 0 | 0 | 23 | 0 | 190 | 0 | 385 |
| | (D150) | 98X002 | 0 | 0 | 0 | 10 | 1 | 123 | 1 | 68 |

At wk 15 (after three injections), the levels of p11c-specific T cells were higher in the D121 cohort compared to the D150 cohort. Moreover, the immune response to DNA in D121 was higher than for DNA in D150 after two injections (see data for wk 6).

In summary, the results in Tables 8A and 8B indicate that the immune response induced by DNA/BAK formulations containing CRL-1005 (formulations D118, D118a and D121) is stronger and appears earlier than the immune response induced by DNA/BAK formulations lacking CRL-1005 (D149, D150) or by DNA/CRL-1005 formulations lacking BAK (D113).

### EXAMPLE 14

DNA/BAK/CRL-1005 Effectively Primes for an Adenovirus Boost.

Experiment Design—Cohorts of 3-5 monkeys were vaccinated with (1) 5 mg V1Jns-gag DNA in PBS (D101) and (2) 5 mg V1Jns-gag+D118a (7.5 mg CRL-1005+0.6 mM BAK). All vaccines were stored frozen in glass vials and a plastic secondary container; just prior to injection, the vials were slowly warmed to room temperature. Immunizations were conducted at 0, 4, 8 weeks. At wk 26, the animals were boosted with 10ˆ7 viral particles of a replication-defective adenovirus type 5 (Ad5) vector that expresses the same HIV-1 gag gene. Peripheral blood mononuclear cells (PBMCs) from the vaccinees were assayed at several time points via elispot assay against an HIV-1 gag peptide pool (gag H).

At 4 and 8 wks after the boost (Table 9), the frequencies of gag-specific T cells were 2-fold higher in the DNA/D118a-primed animals compared to the control cohort. These results indicate that DNA/BAK/CRL-1005 formulations are more effective than DNA in PBS at priming the immune response for an Adenovirus boost.

TABLE 9
Enhancement of Adenovirus Boost by a D118a Priming Series.
Number of SFC per million PBMCs.

| | Vaccination | Boost | | T = 24 | | T = 28 | | T = 30 | | T = 34 | |
|---|---|---|---|---|---|---|---|---|---|---|---|
| Group# | (Formulation Code) | T = 26 Wk | Monkey# | Medium | gag H | Medium | gag H | Medium | gag H | Medium | gag H |
| 1 | DNA in PBS | Ad5gag | CB5H | 6 | 85 | 19 | 956 | 0 | 316 | 1 | 71 |
| | (D101) | 10\hat{ }7 vp | CC6X | 0 | 35 | 3 | 1705 | 1 | 755 | 1 | 396 |
| | | | AW3G | 8 | 65 | 10 | 989 | 0 | 395 | 1 | 154 |
| 2 | DNA + | Ad5gag | AW20 | 9 | 205 | 18 | 565 | 8 | 404 | 4 | 141 |
| | CRL-1005/7.5 mgs + | 10\hat{ }7 vp | CA4R | 1 | 105 | 14 | 1384 | 10 | 978 | 15 | 683 |
| | 0.6 mM BAK | | CB58 | 1 | 208 | 0 | 636 | 1 | 828 | 0 | 1126 |
| | (D118a) | | CB5W | 1 | 62 | 5 | 543 | 1 | 349 | 3 | 83 |
| | | | CB7D | 1 | 759 | 0 | 2278 | 4 | 1831 | 6 | 898 |
| 3 | None | None | 98D201 | 1 | 2 | 3 | 0 | 0 | 0 | 0 | 3 |

### EXAMPLE 15

Stability of Plasmid DNA/CRL1005 Formulations.

The stability of supercoiled plasmid DNA was evaluated in a formulation containing 5.0 mg/mL DNA, 12 mg/mL CRL-1005 and 0.5 mM BAK. Agarose gel electrophoresis of the DNA after 9 months of storage at 2-8° C. indicated that the DNA in the CRL-1005 formulation had 99% of the initial supercoiled content remaining. A control sample of 5.0 mg/mL plasmid DNA in PBS had 98% of the initial supercoiled content remaining (the initial supercoiled content was 91-92% for both samples). These data indicate that CRL-1005, at concentrations up to 12 mg/mL, does not significantly affect the storage stability of plasmid DNA in PBS.

The stability of the CRL-1005 polymer was also examined in the same experiment. The stability of the polymer was evaluated using HPLC gel permeation chromatography (GPC) to determine the percentage of polymer with a molecular weight below 7,000 daltons. An increase in the percentage of low molecular weight polymer over time in storage would indicate polymer degradation. The GPC results indicated that the percentage of low MW polymer (<,7000 daltons) was 16.73% at time zero and 16.1% after 6 months in storage at 2-8° C. (these results are within assay variability; % RSD=5-6%). These results indicate that CRL-1005 is stable for at least 6 months at 2-8° C. in a formulation containing 5.0 mg/mL DNA and 0.45 mM BAK.

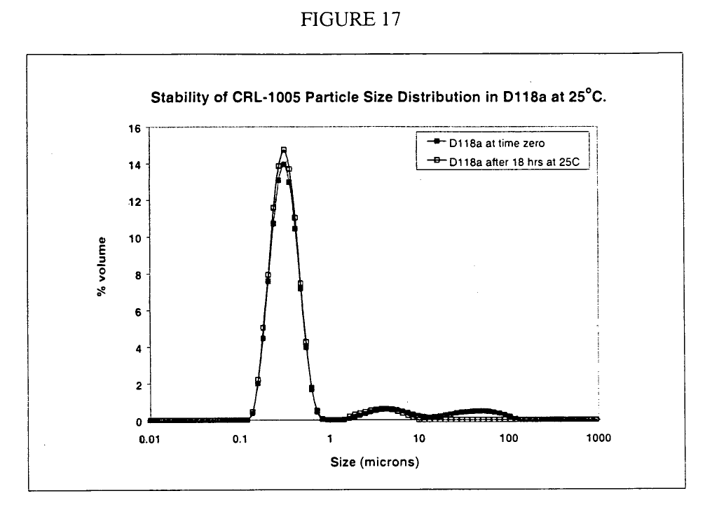

Another important aspect of formulation stability is the stability of the particle size distribution. Since the CRL-1005 is soluble during storage at 5° C., the CRL-1005 particles are not formed until the formulation is taken out of storage and warmed above the cloud point at room temperature, just prior to use. A formulation useful for vaccine or gene therapy use should have a particle size distribution that is stable until administration. A vaccine or gene therapy formulation with a particle size distribution that is stable for at least 8 hours at room temperature would be especially useful. To determine the stability of the particle size distribution a formulation (D118a) containing 5.0 mg/mL DNA, 7.5 mg/mL CRL-1005 and 0.6 mM BAK was prepared. The particle size distribution was determined by static laser diffraction light scattering at time zero and after 18 hours of incubation at 25° C. The results, shown in FIG. 17, indicate that the particle size distribution of CRL-1005 in D118a is stable at room temperature for at least 18 hours after warming above the cloud point.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. An adjuvant, comprising:
a) a block copolymer of the general formula:
HO(C₂H₄O)ₐ(C₃H₆O)_{b}(C₂H₄O)ₐ H;,
wherein (b) represents a number such that the molecular weight of the hydrophobic POP portion (C₃H₆O) is up to approximately 20,000 daltons and wherein (a) represents a number such that the percentage of hydrophilic POE portion (C₂H₄O) is between approximately 1% and 40% by weight; and,
b) a cationic surfactant.

2. The adjuvant of claim 1 wherein subscript (b) of the general formula of the block copolymer represents a number such that the molecular weight of the hydrophobe (C₃H₆O) is between approximately 9000 Daltons and 15,000 Daltons and subscript (a) of the general formula represents a number such that the percentage of hydrophile (C₂H₄O) is between approximately 3% and 35%, preferably at or below 10%.

3. The adjuvant of claim 1 wherein subscript (b) of the general formula of the block copolymer represents a number such that the molecular weight of the hydrophobe (C₃H₆O) is between approximately 9000 Daltons and subscript (a) of the general formula represents a number such that the percentage of hydrophile (C₂H₄O) is between approximately 3% and 5%.

4. The adjuvant of claim 1 wherein subscript (b) of the general formula of the block copolymer represents a number such that the molecular weight of the hydrophobe (C₃H₆O) is between approximately 12000 Daltons and subscript (a) of the general formula represents a number such that the percentage of hydrophile (C₂H₄O) is approximately 5%.

5. The adjuvant of claim 1 wherein the block copolymer is CRL-1005.

6. The adjuvant of claims 1, 2, 3, 4 or 5 wherein the cationic surfactant is selected from the group consisting of benzalkonium chloride, benzethonium chloride, cetramide, cetylpyridinium chloride and cetyl trimethylammonium chloride.

7. A polynucleotide vaccine formulation, comprising:
a) a population of polynucleotide molecules; and,
b) an adjuvant, the adjuvant consisting of a block copolymer of the general formula:
HO(C₂H₄O)ₐ(C₃H₆O)_{b}(C₂H₄O)ₐ H,
wherein (b) represents a number such that the molecular weight of the hydrophobic POP portion (C₃H₆O) is up to approximately 20,000 daltons and wherein (a) represents a number such that the percentage of hydrophilic POE portion (C₂H₄O) is between approximately 1% and 40% by weight; and,
c) a cationic surfactant,
wherein a portion of the polynucleotide vaccine molecule population are associated with the adjuvant and the remaining portion of the polynucleotide vaccine molecule population are free within a pharmaceutically acceptable buffer.

8. The polynucleotide vaccine formulation of claim 7 wherein subscript (b) of the general formula of the block copolymer represents a number such that the molecular weight of the hydrophobe (C₃H₆O) is between approximately 9000 Daltons and 15,000 Daltons and subscript (a) of the general formula of the block copolymer represents a number such that the percentage of hydrophile (C₂H₄O) is between approximately 3% and 35%, preferably at or below 10%.

9. The polynucleotide vaccine formulation of claim 7 wherein subscript (b) of the general formula of the block copolymer represents a number such that the molecular weight of the hydrophobe (C₃H₆O) is between approximately 9000 Daltons and subscript (a) of the general formula of the block copolymer represents a number such that the percentage of hydrophile (C₂H₄O) is between approximately 3% and 5%.

10. The polynucleotide vaccine formulation of claim 7 wherein subscript (b) of the general formula of the block copolymer represents a number such that the molecular weight of the hydrophobe (C₃H₆O) is between approximately 12000 Daltons and subscript (a) of the general formula of the block copolymer represents a number such that the percentage of hydrophile (C₂H₄O) is approximately 5%.

11. The polynucleotide vaccine formulation of claim 7 wherein the block copolymer is CRL-1005.

12. The polynucleotide vaccine formulations of claims 7, 8, 9, 10 or 11 wherein the cationic surfactant is selected from the group consisting of benzalkonium chloride, benzethonium chloride, cetramide, cetylpyridinium chloride and cetyl trimethylammonium chloride.

13. The polynucleotide vaccine formulation of claim 11 which is selected from the group consisting of D118, D118a, and D121.

14. A method of inducing an immune response in an mammalian host which comprises introducing the polynucleotide vaccine formulation of claim 7 into said mammalian host.

15. A method of inducing an immune response in an mammalian host which comprises introducing the polynucleotide vaccine formulation of claim 8 into said mammalian host.

16. A method of inducing an immune response in an mammalian host which comprises introducing the polynucleotide vaccine formulation of claim 9 into said mammalian host.

17. A method of inducing an immune response in an mammalian host which comprises introducing the polynucleotide vaccine formulation of claim 10 into said mammalian host.

18. A method of inducing an immune response in an mammalian host which comprises introducing the polynucleotide vaccine formulation of claim 11 into said mammalian host.

19. A method of inducing an immune response in an mammalian host which comprises introducing the polynucleotide vaccine formulation of claim 12 into said mammalian host.

20. A method of inducing an immune response in an mammalian host which comprises introducing the polynucleotide vaccine formulation of claim 13 into said mammalian host.

21. A polynucleotide vaccine formulation, comprising:
a) a population of polynucleotide molecules; and,
b) an adjuvant, the adjuvant consisting of a block copolymer of the general formula:
HO(C₂H₄O)ₐ(C₃H₆O)_{b}(C₂H₄O)ₐ H,
wherein (b) represents a number such that the molecular weight of the hydrophobic POP portion (C₃H₆O) is up to approximately 20,000 daltons and wherein (a) represents a number such that the percentage of hydrophilic POE portion (C₂H₄O) is between approximately 1% and 40% by weight;
c) a cationic surfactant; and,
d) a non-ionic surfactant
wherein a portion of the polynucleotide vaccine molecule population are associated with the adjuvant and the remaining portion of the polynucleotide vaccine molecule population are free within a pharmaceutically acceptable buffer.

22. The polynucleotide vaccine formulation of claim 21 wherein subscript (b) of the general formula of the block copolymer represents a number such that the molecular weight of the hydrophobe (C₃H₆O) is between approximately 9000 Daltons and 15,000 Daltons and subscript (a) of the general formula of the block copolymer represents a number such that the percentage of hydrophile (C₂H₄O) is between approximately 3% and 35%, preferably at or below 10%.

23. The polynucleotide vaccine formulation of claim 21 wherein subscript (b) of the general formula of the block copolymer represents a number such that the molecular weight of the hydrophobe (C₃H₆O) is between approximately 9000 Daltons and subscript (a) of the general formula of the block copolymer represents a number such that the percentage of hydrophile (C₂H₄O) is between approximately 3% and 5%.

24. The polynucleotide vaccine formulation of claim 21 wherein subscript (b) of the general formula of the block copolymer represents a number such that the molecular weight of the hydrophobe (C₃H₆O) is between approximately 12000 Daltons and subscript (a) of the general formula of the block copolymer represents a number such that the percentage of hydrophile (C₂H₄O) is approximately 5%.

25. The polynucleotide vaccine formulation of claim 21 wherein the block copolymer is CRL-1005.

26. The polynucleotide vaccine formulations of claims 21, 22, 23, 24 or 25 wherein the cationic surfactant is selected from the group consisting of benzalkonium chloride, benzethonium chloride, cetramide, cetylpyridinium chloride and cetyl trimethylammonium chloride.
